(19) United States
(12) Patent Application Publication
(10) Pub. No.: US 2024/0390493 A1
(43) Pub. Date: Nov. 28, 2024

(54) METHODS AND COMPOSITIONS TO AUGMENT EFFICACY AND REDUCE TOXICITY OF NON-ENGRAFTING, CD8-DEPLETED ALLOGENIC DONOR LYMPHOCYTE INFUSIONS

(71) Applicants: The Johns Hopkins University, Baltimore, MD (US); Iyuda Therapeutics, LLC, Owings Mills, MD (US)

(72) Inventors: Ephraim Fuchs, Baltimore, MD (US); Robert E. Tennant, Owings Mills, MD (US)

(21) Appl. No.: 18/695,999

(22) PCT Filed: Sep. 29, 2022
(86) PCT No.: PCT/US2022/045241

§ 371 Date: Mar. 27, 2024

Related U.S. Application Data
(60) Provisional application No. 63/332,616, filed on Apr. 19, 2022.
(60) Provisional application No. 63/287,890, filed on Dec. 9, 2021.
(60) Provisional application No. 63/250,061, filed on Sep. 29, 2021.

(51) Int. Cl. A61K 39/00

(52) U.S. Cl. CPC A61K 39/4611; A61K 39/4621; A61K 39/464462; A61K 2239/49; A61K 2239/55; A61K 2239/59

(57) ABSTRACT

Provided herein are methods and compositions to augment the efficacy and reduce toxicity of non-engrafting, CD8-depeleted allogeneic donor lymphocyte infusions. The compositions comprise isolated leukocytes obtained from a donor subject that (i) are mismatched to a recipient subject for at least one human leukocyte antigen (HLA) Class II allele mismatch in the donor versus recipient (graft-versus-host) direction relative to the recipient subject or (ii) is mismatched to a recipient subject for at least one human leukocyte antigen (HLA) Class II allele mismatch in the donor versus recipient (graft-versus-host) direction relative to the recipient subject, is matched to the recipient for at least one human leukocyte antigen (HLA) Class II allele, and has CD4+ T cell immunity against an antigen present in a recipient subject.

### CROSS-REFERENCE TO RELATED APPLICATIONS

The present application claims the benefit of priority under 35 U.S.C. § 119(e) of U.S. Provisional Application Ser. No. 63/250,061, filed on Sep. 29, 2021; 63/287,890, filed on Dec. 9, 2021; and 63/332,616, filed on Apr. 19, 2022. The disclosures of the prior applications are considered part of and are herein incorporated by reference in the disclosure of this application in their entirety.

### FIELD OF THE INVENTION

This disclosure relates generally to allogeneic lymphocyte compositions and more specifically to use of such compositions in methods to augment the efficacy and reduce the toxicity of non-engrafting, CD8-depleted allogeneic donor lymphocyte infusions.

### BACKGROUND

Cancer immunotherapy has emerged as a promising cancer treatment modality. The goal of cancer immunotherapy is to harness the immune system for selective destruction of cancer cells while leaving normal tissues unharmed.

Cytotoxic CD8+ T cells are a major population of immune cells that control and clear tumor cells. Zhang et al., (2020), Front. Cell Dev. Biol., 8:17. However, T cells are not as effective against cancer as expected. Due to immunologic tolerance and immunosuppressive mechanisms, CD8+ T cells are often sub-optimally primed and for this reason and others can fail to be effective or enter a dysfunctional state known as T cell exhaustion. T cell exhaustion leads to attenuated effector function whereby cytotoxic CD8+ T cells are impaired in their ability to kill cancer cells, leading to tumor progression.

There is great interest in developing methods to revive or replace exhausted T cells to restore the anti-tumor immune response. However, challenges still remain to develop effective methods for reviving exhausted T cells.

Allogeneic T cell therapy is a form of cancer immunotherapy in which lymphocytes are collected from the peripheral blood or bone marrow of healthy donors and transfused into a patient with cancer. This therapy is typically given in the form of allogeneic stem cell transplantation, in which the patient receives highly immunosuppressive conditioning followed by an infusion of a stem cell graft containing unselected populations of mature T cells. The goal of allogeneic stem cell transplantation is to obtain sustained engraftment of the donor cells, but it is associated with significant toxicities including graft-versus-host disease (GVHD), an immunologic attack on normal tissues, and the treatment has not been proven to be effective in the treatment of solid tumor malignancies. International Application No. PCT/US2013/032129 describes an approach termed non-engrafting donor lymphocyte infusion, in which a patient is treated with lymphodepleting chemotherapy followed by a transfusion of peripheral blood cells, depleted of CD8+ cells, from a healthy, partially or fully human leukocyte antigen (HLA)-mismatched donor. The proposed therapeutic mechanism of mismatched, CD8-depleted donor lymphocyte infusion (DLI) is that alloreactive donor CD4+ T cells secrete cytokines and deliver signals through recipient antigen-presenting cells (APCs) to reverse exhaustion in recipient-derived, tumor-specific CD8+ T cells. While this therapy can induce anti-tumor responses against hematologic malignancies, its efficacy against solid tumors is limited, possibly by the reversion of tumor-specific CD8+ T cells to an exhausted state.

As such, there continues to be an unmet need for methods that can efficiently and sustainably awaken exhausted T cells and induce regression of tumors while reducing the risk of sustained engraftment and graft-versus-host disease.

### SUMMARY OF THE INVENTION

This disclosure relates to allogeneic lymphocyte compositions and methods for augmenting the efficacy and reducing the toxicity of non-engrafting, CD8-depleted allogeneic lymphocyte infusions.

The disclosure provides allogeneic lymphocyte compositions that are depleted of CD8+ T cells and contain CD4+ T cells that are modified for enhanced in vivo anti-tumor activity. Without committing to a particular mechanism, the modifications may increase the frequency of type 1, or Tₕ1 CD4+ T cells, which secrete interferon gamma (IFNγ) and provide optimal help for recipient CD8+ T cells or render CD4+ T cells more resistant to exhaustion or suppression of activity. The allogeneic lymphocyte composition can provide a source of CD4+ T cell help to reverse exhaustion of a subject's T cells while donor CD8+ T cell depletion reduces the risk of sustained engraftment and graft-versus-host disease. In addition to providing help to revive exhausted CD8+ T cells, donor Tₕ1 cells can re-program the tumor microenvironment to become immunostimulatory and reverse exhaustion of CD8+ T cells while limiting the toxicities of graft versus host disease.

This disclosure relates to pharmaceutical compositions comprising a plurality of isolated leukocytes that are mismatched to a recipient subject for at least one human leukocyte antigen (HLA) Class II allele in the donor versus recipient (graft-versus-host) direction relative to the recipient subject. The isolated leukocytes can be obtained from a donor subject. The isolated leukocytes can also be obtained from other sources, such as cell lines or cord blood. The leukocytes are depleted of CD8+ T cells by about 10-fold or greater relative to un-depleted leukocytes. The leukocytes can be further modified to suppress Bruton's tyrosine kinase (BTK), interleukin-2-inducible T cell kinase (ITK), delta isoform of phosphoinositide 3-kinase (PI3Kδ), helios, blimp1, SOCS1, GATA3, IL-10, STAT3, TGF-beta Receptor II, LAG-3, PD-1, TNF-alpha, TOX, CD25, foxp3, Ezh2, or combinations thereof.

The disclosure also relates to pharmaceutical compositions comprising a plurality of isolated leukocytes obtained from an allogeneic donor subject. The donor CD4+ T cells have been stimulated in vivo or ex vivo by an antigen present in a recipient subject, and the donor lymphocytes comprise at least one HLA Class II allele match relative to the recipient. Alternatively, the donor CD4+ T cells have been stimulated in vivo or ex vivo by an antigen that is not present in a recipient, the donor lymphocytes comprise at least one HLA Class II allele match relative to the recipient, and both the antigen and the donor CD4+ T cells are introduced into the recipient. The plurality of isolated leukocytes can also be obtained from other sources such as, for example, cell lines or cord blood. The leukocytes are depleted of CD8+ T cells by about 10-fold or greater relative to un-depleted leukocytes. The leukocytes are modified to suppress Bruton's tyrosine kinase (BTK), interleukin-2-inducible T cell kinase (ITK), delta isoform of phosphoinositide 3-kinase (PI3Kδ), helios, blimp1, SOCS1, GATA3, IL-10, STAT3, TOX, CD25, foxp3, Ezh2, TGF-beta Receptor II, LAG-3, PD-1, TNF-alpha, or combinations thereof.

The disclosure also relates to pharmaceutical compositions comprising a plurality of isolated leukocytes that are obtained from a donor subject and (i) are mismatched to a recipient subject for at least one HLA Class II allele in the donor versus recipient (graft-versus-host) direction relative to the recipient subject and (ii) the donor CD4+ T cells have been stimulated in vivo or ex vivo by an antigen present in a recipient subject, and the donor subject comprises at least one human leukocyte HLA Class II allele match relative to the recipient. Alternatively, the donor CD4+ T cells (i) are mismatched to a recipient subject for at least one HLA Class II allele mismatch in the donor versus recipient (graft-versus-host) direction relative to the recipient subject and (ii) the donor CD4+ T cells have been stimulated in vivo or ex vivo by an antigen not present in a recipient subject, and the donor subject comprises at least one human leukocyte HLA Class II allele match relative to the recipient, and (iii) both the antigen and the donor CD4+ T cells are introduced into the recipient. The plurality of isolated leukocytes can also be obtained from other sources such as cell lines or cord blood. The leukocytes are depleted of CD8+ T cells by about 10-fold or greater relative to un-depleted leukocytes. At least a portion of the CD4+ T cells are modified to inhibit the activity of Bruton's tyrosine kinase (BTK), interleukin-2-inducible T cell kinase (JTK), delta isoform of phosphoinositide 3-kinase (PI3Kδ), helios, blimp1, SOCS1, GATA3, IL-10, STAT3, TOX, CD25, foxp3, Ezh2, TGF-beta Receptor II, LAG-3, PD-1, TNF-alpha, or combinations thereof.

The disclosure also relates to methods for producing an allogeneic lymphocyte composition comprised by obtaining a peripheral blood cell composition that (i) is mismatched to a recipient subject for at least one HLA Class II allele in the donor versus recipient (graft-versus-host) direction relative to the recipient subject and (ii) has CD4+ T cell immunity against an antigen present in a recipient subject, and the donor subject comprises at least one HLA Class II allele match relative to the recipient. Alternatively, the donor CD4+ T cells (i) are mismatched to a recipient subject for at least one HLA Class II allele mismatch in the donor versus recipient (graft-versus-host) direction relative to the recipient subject and (ii) the donor CD4+ T cells have been stimulated in vivo or ex vivo by an antigen not present in a recipient subject, and the donor subject comprises at least one human leukocyte HLA Class II allele match relative to the recipient, and (iii) both the antigen and the donor CD4+ T cells are introduced into the recipient. The method further comprises isolating leukocytes from the peripheral blood cell composition and depleting the number of CD8+ T cells in the leukocytes by at least 10-fold or greater relative to un-depleted leukocytes. Next, the method comprises suppressing Bruton's tyrosine kinase (BTK), interleukin-2-inducible T cell kinase (JTK), delta isoform of phosphoinositide 3-kinase (PI3Kδ), helios, blimp1, SOCSI, GATA3, IL-10, STAT3, TOX, CD25, foxp3, Ezh2, TGF-beta Receptor II, LAG-3, PD-1, TNF-alpha, or combinations thereof. The method further comprises a treatment to promote differentiation of at least a portion of T cells toward Tₕ1 CD4+ T cells, maintain CD4+Tₕ1 cells in their state of differentiation, or prevent exhaustion or suppression of the CD4+ T cells. The method can further include culturing the leukocytes in vitro. The method can further comprise culturing the leukocytes ex vivo.

The methods for producing an allogeneic lymphocyte composition further comprise adding one or more cytokines. The one or more cytokines are IL-2, IL-7, IL-12, IL-15, IL-18, IFNγ, or IL-21. The method can further comprise adding one or more antibodies. The one or more antibodies can be an anti-IL3 antibody, an anti-IL-4 antibody, an anti-CD3 antibody, an anti-CD200 antibody or an anti-CD28 antibody.

A portion of the T cells in the pharmaceutical composition can be differentiated to Tₕ1 CD4+ T cells. A portion of the T cells can be biased toward Tₕ1 CD4+ T cell differentiation by inhibition of BTK. The T cells are biased toward Tₕ1 CD4+ T cell differentiation by inhibition of JTK.

The T cells can be biased toward Tₕ1 CD4+ T cell differentiation, maintained in the Tₕ1 differentiation state, or made resistant to exhaustion or suppression of function by inhibition of PI3Kδ. The T cells can be biased toward Tₕ1 CD4+ T cell differentiation, maintained in the Tₕ1 differentiation state, or made resistant to exhaustion or suppression of function by inhibition of Foxp3. The T cells can be biased toward Tₕ1 CD4+ T cell differentiation, maintained in the Tₕ1 differentiation state, or made resistant to exhaustion or suppression of function by inhibition of GATA3. The T cells can be biased toward Tₕ1 CD4+ T cell differentiation, maintained in the Tₕ1 differentiation state, or made resistant to exhaustion or suppression of function by inhibition of STAT3. The T cells can be biased toward Tₕ CD4+ T cell differentiation, maintained in the Tₕ1 differentiation state, or made resistant to exhaustion or suppression of function by inhibition of CD25. The T cells can be biased toward Tₕ1 CD4+ T cell differentiation, maintained in the Tₕ1 differentiation state, or made resistant to exhaustion or suppression of function by inhibition of Ezh2. The T cells can be biased toward Tₕ1 CD4+ T cell differentiation, maintained in the Tₕ1 differentiation state, or made resistant to exhaustion or suppression of function by inhibition of BTK and JTK. The T cells can be biased toward Tₕ1 CD4+ T cell differentiation and against regulatory T cell differentiation by inhibition of both BTK and PI3Kδ. The T cells can be biased toward Tₕ1 CD4+ T cell differentiation by inhibition and against regulatory T cell differentiation of both ITK and PI3Kδ. The T cells are biased toward Tₕ1 CD4+ T cell differentiation by inhibition and against regulatory T cell differentiation of BTK, ITK and PI3Kδ. The T cells can be biased toward Tₕ1 CD4+ T cell differentiation, maintained in the Tₕ1 differentiation state, or made resistant to exhaustion or suppression of function by inhibition of TGF-beta Receptor II. The T cells can be biased toward Tₕ1 CD4+ T cell differentiation, maintained in the Tₕ1 differentiation state, or made resistant to exhaustion or suppression of function by inhibition of LAG-3. The T cells can be biased toward Tₕ1 CD4+ T cell differentiation, maintained in the Tₕ1 differentiation state, or made resistant to exhaustion or suppression of function by inhibition of PD-1.

BTK, ITK and PI3Kδ, helios, blimp1, SOCS1, GATA3, IL-10, STAT3, TOX, CD25, foxp3, TGF-beta Receptor II, LAG-3, PD-1, TNF-alpha, or Ezh2 can be inhibited with an inhibitor or by genetic modification. The BTK inhibitor can be acalabrutinib, zanubrutinib, LFM-A13, dasatinib or AVL-292. In embodiments, BTK inhibitor is not ibrutinib. The ITK inhibitor can be aminothiazole, aminobenzimidazole, indole, pyridine or prn694. The PI3Kδ inhibitor can be idelalisib, copanlisib, duvelisib, umbralisib, ME-4401, RP6503, perifosine, buparlisib, or dactolisib.

The ITK inhibitor, the BTK inhibitor, the PI3Kδ inhibitor, the Helios inhibitor, the Blimp1 inhibitor, the SOCS1 inhibitor, the TGF-beta Receptor II inhibitor, the LAG-3 inhibitor, the PD-1 inhibitor, the TNF-alpha inhibitor, the Foxp3 inhibitor, the GATA3 inhibitor, the IL-10 inhibitor, the STAT3 inhibitor, the CD25 inhibitor, the Ezh2 inhibitor, or the TOX inhibitor can be a small molecule, a small interfering RNA (siRNA), or short hairpin RNA (shRNA).

BTK, ITK, PI3Kδ, Helios, Blimp1, SOCS1, Foxp3, GATA3, IL-10, STAT3, Ezh2, CD25, TGF-beta Receptor II, LAG-3, PD-1, TNF-alpha, or TOX can be inhibited by deleting the BTK gene, the ITK gene, the PI3Kδ gene, the Helios gene, the Blimp1 gene, the SOCS1 gene, the Foxp3 gene, the GATA3 gene, the IL-10 gene, the STAT3 gene, the Ezh2 gene, the CD25 gene, the TGF-beta Receptor II gene, the LAG-3 gene, the PD-1 gene, the TNF-alpha gene, or the TOX gene from the genome. The BTK gene, the ITK gene, the PI3Kδ gene, the Helios gene, the Blimp1 gene, the SOCS1 gene, the Foxp3 gene, the GATA3 gene, the IL-10 gene, the STAT3 gene, the Ezh2 gene, the CD25 gene, the TGF-beta Receptor II gene, the LAG-3 gene, the PD-1 gene, the TNF-alpha gene, or the TOX gene can be deleted from the genome using CRISPR or TALEN.

Differentiation of the T cells into T regulatory cells can be attenuated with an inhibitor or by genetic modification. Differentiation of the T cells into T regulatory cells can be attenuated through inhibition of PI3Kδ, Foxp3, CD25, TGF-beta Receptor II, LAG-3, PD-1, or Ezh2. PI3Kδ, Foxp3, CD25, TGF-beta Receptor II, LAG-3, PD-1, or Ezh2 can be inhibited with a PI3Kδ inhibitor, a Foxp3 inhibitor, a CD25 inhibitor, a TGF-beta Receptor II inhibitor, a LAG-3 inhibitor, a PD-1 inhibitor, or an Ezh2 inhibitor or by genetic modification. The PI3Kδ inhibitor can be idelalisib, copanlisib, duvelisib, umbralisib, ME-4401, RP6503, perifosine, buparlisib, or dactolisib. The genetic modification can comprise deletion of the PI3Kδ gene, the Foxp3 gene, the CD25 gene, or the Ezh2 gene.

Differentiation of the T cells into T regulatory cells or the function of regulatory T cells can be attenuated with a small molecule, a small interfering RNA (siRNA), or short hairpin RNA (shRNA) against BTK, ITK, PI3Kδ, Helios, Blimp1, SOCS1, Foxp3, GATA3, IL-10, STAT3, Ezh2, CD25, TGF-beta Receptor II, LAG-3, PD-1 or TOX. Differentiation of the T cells into T regulatory cells or the function of T regulatory cells can be attenuated by modifying the isolated leukocytes obtained from the donor subject to express a dominant negative transforming growth factor-beta RII receptor.

Differentiation of the T cells into Tₕ2 cells or function as Tₕ2 cells can be attenuated with an inhibitor or by genetic modification. Differentiation of the T cells into Tₕ2 cells or function as Tₕ2 cells can be attenuated through inhibition of GATA3. Differentiation of the T cells into Th17 cells or function as Th17 cells can be attenuated through inhibition of STAT3.

The HLA Class II match can be an HLA-DRB1 allele, an HLA-DQB1 allele, or an HLA-DPB1 allele.

Activation of myeloid cells in the pharmaceutical composition can be inhibited. Activation of myeloid cells can be inhibited through inhibition of BTK, ITK, PI3Kδ, Helios, Blimp1, SOCS1, Foxp3, GATA3, IL-10, STAT3, TGF-beta Receptor II, LAG-3, PD-1, TNF-alpha, or TOX. BTK can be inhibited in CD4+ T cells by at least about 50% relative to BTK basal activity. ITK in CD4+ T cells can be inhibited by at least about 50% relative to ITK basal activity. PI3Kδ can be inhibited by at least about 50% relative to PI3Kδ basal activity. The amount of ITK, BTK, or PI3Kδ inhibited can be measured by Western Blot.

The expression of IFNγ or IL-12 may be increased by at least about 10-fold relative to leukocytes that are not modified to suppress BTK, ITK, PI3Kδ, Helios, Blimp1, SOCS1, Foxp3, GATA3, IL-10, STAT3, CD25, Ezh2, TGF-beta Receptor II, LAG-3, PD-1, TNF-alpha, or TOX. The expression of TGFβ or IL-10 is decreased by at least about 10-fold relative to leukocytes that are not modified to suppress BTK, ITK, PI3Kδ, Helios, Blimp1, SOCS1, Foxp3, GATA3, IL-10, STAT3, CD25, Exh2, TGF-beta Receptor II, LAG-3, PD-1, TNF-alpha, or TOX.

The amount of IFNγ, IL-12, TGFβ or IL-10 can be measured by intracellular cytokine staining or ELISA.

The expression of IFNγ or IL-12 can be increased by at least about 10 fold relative to leukocytes that are not modified to suppress BTK, ITK, PI3Kδ, Helios, Blimp1, SOCS1, Foxp3, GATA3, IL-10, STAT3, TOX, TGF-beta Receptor II, LAG-3, PD-1, TNF-alpha, or combinations thereof. The expression of TGFβ or IL-10 can be decreased by at least about 10-fold relative to leukocytes that are not modified to suppress BTK, ITK, PI3Kδ, Helios, Blimp1, SOCS1, Foxp3, GATA3, IL-10, STAT3, TOX, TGF-beta Receptor II, LAG-3, PD-1, TNF-alpha, or combinations thereof.

Differentiation of the T cells into Tₕ2 cells or the function of the T cells as Tₕ2 cells can be attenuated with an inhibitor or by genetic modification. Differentiation of the T cells into Tₕ2 cells or the function of the T cells as Tₕ2 cells can be attenuated through inhibition of GATA3.

Differentiation of the T cells into Th17 cells or the function of the T cells as Th17 cells can be attenuated through inhibition of STAT3. T cells can be biased toward Tₕ1 CD4+ T cell differentiation or function by inhibition of Foxp3. T cells can be biased toward Tₕ1 CD4+ T cell differentiation or function by inhibition of GATA3. T cells can be biased toward Tₕ1 CD4+ T cell differentiation or function by inhibition of STAT3. T cells can be biased toward Tₕ1 CD4+ T cell differentiation or function by inhibition of CD25. T cells can be biased toward Tₕ1 CD4+ T cell differentiation or function by inhibition of Ezh2.

The disclosure further relates to methods of treating cancer, comprising administering to a subject in need thereof an effective amount of (i) a lymphodepleting agent and (ii) the pharmaceutical composition disclosed herein.

Disclosed herein are methods for treating cancer, comprising administering to a subject in need thereof an effective amount of (i) a lymphodepleting agent, (ii) an inhibitor of NLR family pyrin domain containing 3 (NLRP3), and (iii) the pharmaceutical composition disclosed herein.

Disclosed herein are methods for treating cancer, comprising administering to a subject in need thereof an effective amount of (i) a lymphodepleting agent, (ii) an agent that inhibits differentiation of the T cells into T regulatory cells or inhibits the function of T regulatory cells, and (iii) the pharmaceutical composition disclosed herein.

Disclosed herein are methods of potentiating anti-tumor immunity in a subject having a cancer comprising administering to a subject in need thereof an effective amount of (i) a lymphodepleting agent, and (ii) the pharmaceutical composition disclosed herein.

The lymphodepleting agent can be a cytoreductive agent. The cytoreductive agent can be an alkylating agent, an alkyl sulphonate, a nitrosourea, a triazene, an antimetabolite, a pyrimidine analog, a purine analog, a vinca alkaloid, an epipodophyllotoxin, an antibiotic, dibromomannitol, deoxyspergualine, dimethyl myleran or thiotepa. The alkylating agent can be cyclophosphamide. The purine analog can be fluarabine, cladribine, or pentostatin. The cancer may be a hematological cancer. The cancer may be a solid cancer. The hematological cancer can be a leukemia, lymphoma, multiple myeloma, myelodysplastic syndrome, or myeloproliferative disorder.

The leukemia, lymphoma, multiple myeloma, myelodysplastic syndrome or myeloproliferative disorder can be non-Hodgkin lymphoma, chronic lymphocytic leukemia, small lymphocytic lymphoma, diffuse large B-cell lymphoma, follicular lymphoma, marginal zone lymphoma, mantle cell lymphoma, acute lymphoblastic leukemia, acute myeloid leukemia, hairy cell leukemia, AIDS-related lymphoma, cutaneous T cell lymphoma, Hodgkin lymphoma, mycosis fungoides, primary central nervous system lymphoma. Sezary syndrome, T cell lymphoma, Waldenstrom's macroglobulinemia, chronic myeloid leukemia, chronic myelomonocytic leukemia, polycythemia vera, essential thrombocythemia, or idiopathic myelofibrosis.

The solid cancer can be sarcoma, carcinoma, a neurofibromatoma, a colon cancer, a lung cancer, an ovarian cancer, pancreatic cancer, or a breast cancer.

The methods disclosed herein can further comprise administering to the subject in need thereof an additional therapeutic agent. The additional therapeutic agent is a chemotherapeutic agent, radiation therapy, an immunotherapeutic agent, a T cell agonist cytokine, a CAR-T, a CAR-NK, natural killer cells, gamma-delta T cells, antibody-drug conjugate, an antibody, a bispecific or trispecific T cell or NK cell engager, an immune checkpoint inhibitor, small molecule inhibitor, or an oncolytic virus therapy, or a vaccine.

The antibody can be rituximab, Obinutuzumab, ofatumumab, cetuximab, trastuzumab, pertuzumab, brentuximab vedotin, gemtuzumab, trastuzumab emtansine, inotuzumab ozogamicin, glembatumumab vedotin, lorvotuzumab mertansine, cantuzumab mertansine, or milatuzumab-doxorubicin.

The immune checkpoint inhibitor can be an inhibitor of or antibody against PD-Li, PD-1, CTLA-4, LAG-3, TIGIT, or TIM-3.

The small molecule inhibitor can be dasatinib, nilotinib, ponatinib, imatinib, bosutinib, asciminib, lapatinib, or vismodegib.

The pharmaceutical composition can be administered after the lymphodepleting agent.

### BRIEF DESCRIPTION OF DRAWINGS

FIG. 1 is a schematic diagram depicting the study protocol MHC-haploidentical donors vaccinated against a tumor antigen CD4+ T cells having gene deletions.

FIGS. 2A-2D are graphs showing tumor-free survival percentage over days after tumor inoculation in MHC-haploidentical C57BL/6×C3H (B6C3 F1) mice. FIG. 2A shows tumor-free survival of TC1-luci bearing recipients of unvaccinated (squares) versus vaccinated (circles) donor CD4+ T cells. FIG. 2B shows tumor free survival of pBI-11 vaccinated donors cells with nucleofected with Cas9 reagent (squares) versus without Cas9 nucleofection (circles). FIG. 2C shows tumor free survival of unvaccinated donor CD4+ T cells (triangles), pBI-11 vaccinated and Cas9 nucleofected donor CD4+ T cells (squares), and pBI-11 vaccinated donor cells with ITK gene deletion (circles). FIG. 2D shows tumor free survival of pBI-11 vaccinated donor cells with Foxp3 gene deletion (open squares), transforming growth factor-beta receptor type 2 (TGFBR2) gene deletion (open circles), SOCS1 gene deletion (circles), ITK gene deletion (squares), or PD1 gene deletion (triangles).

### DETAILED DESCRIPTION

This disclosure relates to allogeneic lymphocyte compositions and methods for augmenting the efficacy and reducing the toxicity of non-engrafting, CD8-depleted allogeneic lymphocyte infusions.

The inventors have developed allogeneic lymphocyte compositions that are enriched for CD4+Tₕ1 cells and depleted of CD8+ T cells. Without being bound by theory or mechanism, the inventors believe that the allogeneic lymphocyte composition can provide a source of CD4+ T cells that can provide signals to decrease immune suppression, and/or increase immune system activation, and/or reverse the exhaustion of CD8+ T cells upon transfusion and revive the endogenous anti-tumor response while simultaneously minimizing the risk of sustained engraftment and graft-versus-host disease.

The allogeneic lymphocyte compositions disclosed herein comprise a plurality of isolated leukocytes which can be obtained from a donor subject or another source such as a cord blood or a cell line. The leukocytes can be mismatched to a recipient subject for at least one human leukocyte antigen (HLA) Class II allele in the donor versus recipient (graft-versus-host) direction relative to the recipient subject. Alternatively, the donor can comprise at least one HLA class II allele mismatch relative to the recipient in the donor versus the recipient (graft-versus-host) direction and at least one HLA Class II allele match relative to the recipient. The HLA class II allele mismatch or match can be at HLA-DRB1, HLA-DQB1, or HLA-DPB1. For the purposes of this therapy, low expression HLA Class II molecules, for example, HLA-DPA1, HLA-DQA1 and HLA-DRB3, -DRB4, and -DRB5 may not be considered. In some instances the allogeneic composition disclosed herein can comprise a plurality of isolated leukocytes obtained from a cell line or cord blood and modified, or not modified to target specific targets. The cell line can be HLA class II allele matched, partially-matched, or mismatched to the subject.

The leukocytes can be depleted of CD8+ T cells by about 10-fold or greater relative to undepleted leukocytes. The leukocytes can be further modified to suppress Bruton tyrosine kinase (BTK), Interleukin-2-inducible T cell kinase (ITK), phosphatidyl inositol 3-kinase delta isoform (PI3Kδ), helios, blimp1, SOCS1, GATA3, IL-10, STAT3, TOX, CD25, foxp3, Ezh2, TGF-beta Receptor II, LAG-3, PD-1, TNF-alpha, or combinations thereof. Without wishing to be bound by theory or mechanism, the inventors believe that suppression of BTK, ITK, PI3Kδ, helios, blimp1, SOCS1, GATA3, IL-10, STAT3, TOX, CD25, foxp3, Ezh2, TGF-beta Receptor II, LAG-3, PD-1, TNF-alpha, or combinations thereof may promote naïve CD4+ T cells to differentiate to a state, such as type 1 (Th1) CD4+ T cells, that is favorable for helping effector cells of anti-tumor or anti-viral immunity, or prevent post-naïve CD4+ T cells from converting to cells with suboptimal helper activity for anti-tumor or anti-viral immunity. For example, a portion of the T cells may be preferentially differentiated to a CD4+ T cell sub-type (e.g. Th1). Suppression of BTK, ITK, PI3Kδ, helios, blimp1, SOCS1, GATA3, IL-10, STAT3, TOX, CD25, foxp3, Ezh2, TGF-beta Receptor II, LAG-3, PD-1, TNF-alpha, or combinations thereof may also prevent naïve CD4+ T cells from differentiating to states, such as Th2, Th17, or regulatory T cell, that are suboptimal for promoting anti-tumor or anti-viral immunity, or may prevent CD4+ T cells from becoming exhausted or suppressed by other cells from mediating anti-tumor or anti-viral activity.

Certain illustrative and preferred embodiments are described in further detail herein. The embodiments within the specification should not be construed to limit the scope of the disclosure.

### A. Allogeneic Lymphocyte Composition

The disclosure relates to allogeneic lymphocyte compositions. The allogeneic lymphocyte compositions comprise isolated leukocytes obtained from a donor. The donor can comprise at least one HLA class II allele mismatch relative to the recipient in the donor versus the recipient (graft-versus-host, or GVH) direction. The HLA class II allele mismatch can be at HLA-DRB1, HLA-DQB1, or HLA-DPB1.

Donors that have not been vaccinated against one or more tumor-specific antigens, including neoantigens, generally have a low frequency of tumor-specific CD4+ T cells. When using a donor whose immune system has not been vaccinated against one or more tumor-specific antigens, no HLA Class II matching is required between the donor and the recipient because the ability to revive endogenous anti-tumor immunity is based on the activity of alloreactive CD4+ T cells.

Some degree of HLA Class II allele matching is required when vaccinating the donor against tumor-specific antigens or when expanding tumor-specific CD4+ T cells ex vivo since the expanded tumor-specific CD4+ T cells are restricted to donor HLA Class II molecules and are predicted to be ineffective at delivering help in the recipient unless the recipient expresses at least one HLA Class II molecule that is shared by the donor. Among HLA Class II molecules, the preferred molecules for sharing are the high expression molecules HLA-DRB1>HLA-DPB1>HLA-DQB1>>>>HLADRB3,4,5=HLA-DQA1.

The donor can be partially or completely mismatched at HLA class II alleles in the donor anti-recipient (GVH) direction, for example HLA-DRB1, HLA-DQB1, and HLA-DPB1. The donor can be partially or completely mismatched at HLA class II alleles, for example HLA-DRB1, HLA-DQB1, and HLA-DPB1 and completely matched for Class I alleles. The donor can be completely mismatched with unshared HLAs of first-degree relatives of the recipient who are potential donors for allogeneic stem cell transplantation.

The donor leukocytes may be stimulated in vivo or ex vivo to increase the frequency, compared to the unstimulated leukocytes, of CD4+ T cells that proliferate and/or secrete IFNγ in response to a tumor or viral antigen. The stimulation may consist of deliberate in vivo vaccination of the donor against a tumor antigen or a viral antigen. Alternatively, or in addition, donor leukocytes containing CD4+ T cells can be stimulated ex vivo using antigen-presenting cells (APCs), such as dendritic cells, pulsed with a tumor or viral antigen in the presence or absence of CD4+ T cell-polarizing cytokines. The tumor antigen or viral antigen can be present in the recipient. In instances in which the donor is immunized or donor cells are stimulated ex vivo with antigen pulsed-APCs, the donor must comprise at least one HLA Class II allele match relative to the recipient. The HLA class II allele match can be at HLA-DRB1, HLA-DQB1, or HLA-DPBL. In embodiments, the immunized donor can have at least one HLA class II allele mismatch relative to the recipient in the donor versus the recipient (graft-versus-host) direction and at least one HLA Class II allele match relative to the recipient. When the donor leukocytes have not been stimulated to increase the frequency of tumor- or virus-specific CD4+ T cells, the donor HLA Class II molecules HLA-DRB1, HLA-DQB1, and HLA-DPB1 may be fully mismatched to the recipient in the donor anti-recipient (GVH) direction.

In embodiments, the allogeneic lymphocyte composition can comprise leukocytes obtained from a cell line or from blood cord.

A donor sample can be obtained from a cord blood bank. When the donor sample is obtained from a cord blood bank, a desirable sample may include non-frequent and/or rare HLA alleles as a subject is less likely to contain serum antibodies to non-frequent and/or rare HLA allele types. Exemplary rare alleles include, but are not limited to, A*24:41, B*07:02:28, B*35:03:03, B*39:40V, DRB1*13:23, DRB1*14:111 B*44:16 and DRB1*01:31,C*06:49N, B*37:03N, A*24:312N, and A*30:76N.

In embodiments, the recipient may not have detectable antibodies reactive against HLA of the donor. Detectable antibodies can be determined using conventional methods known to those of skill in the art. For example, the recipient may not have antibodies against donor HLA molecules that are detectable by complement-dependent cytotoxicity, in flow cytometric cross-match assays as a positive result is undesirable, or mean fluorescence intensity (MFI) of 3000 or greater in a solid phase immunoassay is unacceptable.

The number of natural killer cells in the allogeneic composition can be less than or equal to the number of natural killer cells in the peripheral blood composition.

In embodiments, the CD4+ T cells present in the compositions are not activated ex vivo.

The leukocytes present in the allogenic composition are depleted of CD8+ T cells. CD8+ T cells can be depleted using any known methods. For example, magnetic bead cell sorters or flow cytometry may be used to deplete the CD8+ T cells. Reducing CD8+ T cells can involve using an anti-CD8+ antibody associated with a magnetic particle or an anti-CD8+ antibody plus complement.

The leukocytes can be depleted of CD8+ T cells by about 1 fold, about 2 fold, about 3 fold, about 4 fold, about 5 fold, about 6 fold, about 7 fold, about 8 fold, about 9 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 200 fold, about 300 fold, about 400 fold, about 500 fold, about 600 fold, about 700 fold, about 800 fold, about 900 fold, about 1,000 fold or greater relative to undepleted leukocytes.

The leukocytes of the allogeneic lymphocyte compositions disclosed herein are further modified to suppress the activity of BTK, ITK, or PI3Kδ, helios, blimp1, SOCS1, GATA3, IL-10, STAT3, TOX, CD25, foxp3, Ezh2, TGF-beta Receptor II, LAG-3, PD-1, TNF-alpha, or combinations thereof. Without wishing to be bound by theory or mechanism, the inventors believe that suppression of BTK, ITK, PI3Kδ, helios, blimp1, SOCS1, GATA3, IL-10, STAT3, TOX, CD25, foxp3, Ezh2, TGF-beta Receptor II, LAG-3, PD-1, TNF-alpha, or combinations thereof may promote naïve CD4+ T cells to differentiate to a state, such as type 1 (Th1) CD4+ T cells, that is favorable for helping effector cells of anti-tumor or anti-viral immunity, or prevent post-naïve CD4+ T cells from converting to cells with suboptimal helper activity for anti-tumor or anti-viral immunity. For example, a portion of the T cells may be preferentially differentiated to a CD4+ T cell sub-type, such as Th1. Alternatively, differentiation of T cells into another CD4+ T cell subtype (e.g., Tₕ2 or Treg) can be suppressed. Without wishing to be bound by theory or mechanism the inventors also believe that suppression of BTK, ITK, PI3Kδ, helios, blimp1, SOCS1, GATA3, IL-10, STAT3, TOX, CD25, foxp3, Ezh2, TGF-beta Receptor II, LAG-3, PD-1, TNF-alpha, or combinations thereof in leukocytes (e.g., donor leukocytes) augments their efficacy and may reduce the toxicity of non-engrafting allogenic donor lymphocyte infusions.

CD4+ T cells are T lymphocytes that express T cell receptors recognizing peptide antigens presented in the context of Class II major histocompatibility complex (MHC II) molecules. Tay et. al. (2021), Cancer Gene Therapy, 28:5-17. CD4+ T cells can differentiate into one of several diverse functional subtypes in response to context-dependent signals, which in turn allows them to provide ‘help’ to appropriate effector immune cells in their primary role as central coordinators of the immune response. CD4+ T cells primarily mediate anti-tumor immunity by providing help for CD8+ T cells and antibody responses, by inducing tumoricidal capacity of macrophages, by secretion of effector cytokines such as IFNγ and tumor necrosis factor-α (TNFα), and, under specific contexts, via direct cytotoxicity against tumor cells.

CD4+ T cells can differentiate into Tₕ1 cells that express IFNγ and TNFα, Th2 cells that express IL-4, IL-5, and IL-13; Th9 cells that express IL-9 and IL-21; Th17 cells that expresses IL-17; TFH cells that express IL-6 and IL-21; and Treg cells that express TGFβ and IL-10. Tay et. al. (2021), Cancer Gene Therapy, 28:5-17.

The leukocyte compositions disclosed herein can comprise tumor infiltrating lymphocytes (“TILs”), chimeric receptor T cells (“CAR-T”), or T-cell receptor (“TCR”)-transduced T cells. For example, the leukocyte composition can comprise TILs. For example, the leukocyte composition can comprise CAR-T cells. For example, the leukocyte composition can comprise TCR-transduced T cells.

As disclosed above, the leukocytes of the allogeneic lymphocyte compositions disclosed herein are modified to inhibit BTK, ITK, or PI3Kδ, helios, blimp1, SOCS1, GATA3, IL-10, STAT3, TOX, CD25, foxp3, Ezh2, TGF-beta Receptor II, LAG-3, PD-1, TNF-alpha, or combinations thereof. The BTK pathway can be inhibited alone or with ITK, or PI3Kδ, or combinations thereof. The ITK pathway can be inhibited alone or with BTK, or PI3Kδ, or combinations thereof. The PI3Kδ pathway can be inhibited alone or with BTK, or ITK, or combinations thereof. In some instances, each of BTK, ITK, or PI3Kδ pathways are inhibited. Helios can be inhibited alone or in combination with BTK, ITK, or PI3Kδ, Blimp1, SOCS1, Foxp3, GATA3, IL-10, STAT3, CD25, Ezh2, TGF-beta Receptor II, LAG-3, PD-1, TNF-alpha, or TOX. Blimp1 can be inhibited alone or in combination with BTK, ITK, PI3Kδ, Helios, Foxp3, GATA3, IL-10, STAT3, CD25, Ezh2, SOCS1, TGF-beta Receptor II, LAG-3, PD-1, TNF-alpha or TOX. SOCS1 can be inhibited alone or in combination with BTK, ITK, PI3Kδ, Blimp1, Helios, Foxp3, GATA3, IL-10, STAT3, CD25, Ezh2, TGF-beta Receptor II, LAG-3, PD-1, TNF-alpha, or TOX. TGF-beta Receptor II can be inhibited alone or in combination with BTK, ITK, PI3Kδ, Blimp1, Helios, Foxp3, GATA3, IL-10, STAT3, CD25, Ezh2, SOCS1, LAG-3, PD-1, TNF-alpha, or TOX. LAG-3 can be inhibited alone or in combination with BTK, ITK, PI3Kδ, Blimp1, Helios, Foxp3, GATA3, IL-10, STAT3, CD25, Ezh2, TGF-beta Receptor II, SOCS1, PD-1, TNF-alpha, or TOX. PD-1 can be inhibited alone or in combination with BTK, ITK, PI3Kδ, Blimp1, Helios, Foxp3, GATA3, IL-10, STAT3, CD25, Ezh2, TGF-beta Receptor II, LAG-3, SOCS1, TNF-alpha, or TOX. TNF-alpha can be inhibited alone or in combination with BTK, ITK, PI3Kδ, Blimp1, Helios, Foxp3, GATA3, IL-10, STAT3, CD25, Ezh2, TGF-beta Receptor II, LAG-3, PD-1, SOCS1, or TOX. Foxp3 can be inhibited alone or in combination with BTK, ITK, PI3Kδ, Helios, Blimp1, SOCS1, Foxp3, GATA3, IL-10, STAT3, CD25, TGF-beta Receptor II, LAG-3, PD-1, TNF-alpha, Ezh2, or TOX. GATA3 can be inhibited alone or with BTK, ITK, or PI3Kδ, Helios, Blimp1, SOCS1, GATA3, IL-10, STAT3, CD25, TGF-beta Receptor II, LAG-3, PD-1, TNF-alpha, Ezh2, or TOX. GATA3 can be inhibited alone or with BTK, ITK, or PI3Kδ, Helios, Blimp1, SOCS1, Foxp3, IL-10, STAT3, CD25, Ezh2, TGF-beta Receptor II, LAG-3, PD-1, TNF-alpha, or TOX. IL-10 can be inhibited alone or with BTK, ITK, PI3Kδ, Helios, Blimp1, SOCS1, Foxp3, GATA3, TGF-beta Receptor II, LAG-3, PD-1, TNF-alpha, STAT3, or TOX. STAT3 can be inhibited alone or in combination with BTK, ITK, or PI3Kδ, Helios, Blimp1, SOCS1, Foxp3, GATA3, IL-10, CD25, TGF-beta Receptor II, LAG-3, PD-1, TNF-alpha, Ezh2, or TOX. TOX can be inhibited alone or with BTK, ITK, PI3Kδ, Helios, Blimp1, SOCS1, Foxp3, GATA3, IL-10, CD25, TGF-beta Receptor II, LAG-3, PD-1, TNF-alpha, Ezh2, or STAT3. CD25 can be inhibited alone or with BTK, ITK, or PI3Kδ, Helios, Blimp1, SOCS1, Foxp3, IL-10, STAT3, GATA3, Ezh2, TGF-beta Receptor II, LAG-3, PD-1, TNF-alpha, or TOX. IL-10 can be inhibited alone or with BTK, ITK, or PI3Kδ, Helios, Blimp1, SOCS1, Foxp3, GATA3, STAT3, TGF-beta Receptor II, LAG-3, PD-1, TNF-alpha, or TOX. Ezh2 can be inhibited alone or with BTK, ITK, PI3Kδ, Helios, Blimp1, SOCS1, Foxp3, IL-10, STAT3, CD25, GATA3, TGF-beta Receptor II, LAG-3, PD-1, TNF-alpha, or TOX. IL-10 can be inhibited alone or with BTK, ITK, or PI3Kδ, Helios, Blimp1, SOCS1, Foxp3, GATA3, STAT3, TGF-beta Receptor II, LAG-3, PD-1, TNF-alpha, or TOX.

The allogeneic lymphocyte compositions can be modified to inhibit BTK. The allogeneic lymphocyte compositions can be modified to inhibit ITK. The allogeneic lymphocyte compositions can be modified to inhibit PI3Kδ. The allogeneic lymphocyte compositions can be modified to inhibit BTK and PI3Kδ. The allogeneic lymphocyte compositions can be modified to inhibit BTK and ITK. The allogeneic lymphocyte compositions can be modified to inhibit ITK and PI3Kδ. The allogeneic lymphocyte compositions can be modified to inhibit BTK, ITK, and PI3Kδ. The allogenic lymphocyte compositions can be modified to inhibit helios. The allogenic lymphocyte compositions can be modified to inhibit blimp1. The allogenic lymphocyte compositions can be modified to inhibit SOCS1. The allogenic lymphocyte compositions can be modified to inhibit TGF-beta Receptor II. The allogenic lymphocyte compositions can be modified to inhibit LAG-3. The allogenic lymphocyte compositions can be modified to inhibit PD-1. The allogenic lymphocyte compositions can be modified to inhibit TNF-alpha. The allogenic lymphocyte compositions can be modified to inhibit GATA3. The allogenic lymphocyte compositions can be modified to inhibit IL-10. The allogenic lymphocyte compositions can be modified to inhibit STAT3. The allogenic lymphocyte compositions can be modified to inhibit TOX. The allogenic lymphocyte compositions can be modified to inhibit CD25. The allogenic lymphocyte compositions can be modified to inhibit foxp3. The allogenic lymphocyte compositions can be modified to inhibit Ezh2.

The allogenic lymphocyte compositions can be modified to attenuate differentiation of CD4+ T cells into T regulatory (Treg) cells or inhibit CD4+ Treg function. Suppression of PI3Kδ can attenuate differentiation into Treg cells or Treg function. Suppression of Foxp3 can attenuate differentiation into Treg cells. Suppression of CD25 can attenuate differentiation into Treg cells. Suppression of Ezh2 can attenuate differentiation into Treg cells.

Differentiation of the T cells into Treg cells can be attenuated by modifying the isolated leukocytes to express a dominant negative transforming growth factor-beta RII receptor. Liu et al., Nature, 2020, 587(7832):115-120.

BTK, ITK, or PI3Kδ, helios, blimp1, SOCS1, GATA3, IL-10, STAT3, TOX, CD25, TGF-beta Receptor II, LAG-3, PD-1, TNF-alpha, foxp3, Ezh2, or combinations thereof can be suppressed (e.g., inhibited) with a pharmacological agent. Alternatively, BTK, JTK, or PI3Kδ, helios, blimp1, SOCS1, GATA3, IL-10, STAT3, TOX, CD25, TGF-beta Receptor II, LAG-3, PD-1, TNF-alpha, foxp3, Ezh2, or combinations thereof can be inhibited by genetic modification.

Th1-mediated events can contribute to toxicities of immunotherapies including cytokine release syndrome (Imus et al., Biol Blood Marrow Transplant, (2019), 25(12):2431-2437) or liver toxicity (Guan et al., Cell Death Dis., (2021), 12(5):431.) via the effects on innate immune cells, such as macrophages and neutrophils. Without wishing to be bound by theory or mechanism, inhibition of BTK, JTK, helios, blimp1, SOCS1, GATA3, IL-10, STAT3, TOX, CD25, foxp3, Ezh2, TGF-beta Receptor II, LAG-3, PD-1, TNF-alpha, or combinations thereof may also reduce or prevent cytokine release syndrome. Suppression of BTK, ITK, helios, blimp1, SOCS1, GATA3, IL-10, STAT3, TOX, CD25, foxp3, Ezh2, TGF-beta Receptor II, LAG-3, PD-1, TNF-alpha, or combinations thereof my reduce or prevent cytokine release syndrome through inhibition of myeloid cell activation.

CD4+ T cells in the leukocytes can be measured using conventional methodologies known by those skilled in the art, for example, flow cytometry. The expression level of cytokines expressed by the CD4+ T cells can be measured using conventional methodologies by those skilled in the art, for example ELISA or intracellular cytokine staining followed by cell surface staining and flow cytometry.

i. BTK

BTK is a member of the Tec family of non-receptor tyrosine kinases, which consists of a PH domain, a TH domain, an SH3 domain, an SH2 domain, and a catalytic domain. BTK is involved in the signaling of multiple receptors including growth factor receptors, cytokine receptors, G-protein coupled receptors, antigen receptors and integrins. BTK in turn activates many of the major downstream signaling pathways that control cell migration, adhesion, survival and proliferation.

BTK can be suppressed (i.e., inhibited) with a pharmacological agent or by genetic modification. The pharmacological agent could be an inhibitor of BTK. Suitable inhibitors of BTK include, but are not limited to, acalabrutinib, zanubrutinib, tirabrutinib, evobrutinib, tolebrutinib, rilzabrutinib, remibrutinib, tirabrutinib, branebrutinib, orelabrutinib, BIIB091, AC0058, PRN473LFM-A13, dasatinib, GD-4059, or AVL-292. The inhibitor can be a small molecule, a small interfering RNA (siRNA), or short hairpin RNA (shRNA). In embodiments, the BTK inhibitor used herein is not ibrutinib. The BTK inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

TK can be suppressed by deleting, or “knocking out” the BTK gene from the genome. Techniques for knocking out genes are known by those skilled in the art. Gene knock-out methods in the art include, but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the BTK gene. A loss of function mutation can help to suppress gene function by creating a mutation in the BTK gene. Gene editing techniques that can be employed to suppress BTK include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress BTK. A mutation can be made in one or more of the protein domains. For example, a mutation can be made in the pleckstrin homology domain, the proline-rich TEC homology (TH) domain, or the SRC homology domains (SH2 or SH3).

Suppression of BTK can decrease the number or frequency of Th2-polarized T cells in the leukocytes. Suppression of BTK can increase the number or frequency of Th1-polarized T cells in the leukocytes. Suppression of BTK can promote differentiation of T cells to Th1. Suppression of BTK can decrease IL-10, IL-4, or IL-13 expression in the leukocytes. Suppression of BTK can increase IFN-7 expression in the leukocytes. Suppression of BTK can increase IL-12 expression in the leukocytes.

Suppression of BTK can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where BTK is not suppressed. Suppression of BTK can decrease the population of Th2 by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where BTK is not suppressed.

Suppression of BTK can increase the expression of one or more Th1 cell-related markers. Suppression of BTK can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where BTK is not suppressed. The one or more Th1 related markers can include CCR1, CD4, CD26, CD94, CD119, CD183, CD195, CD212, GM-CSF, Granzyme B, IFN-α, IFN-7, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-7, IL-2, IL-12 or any combination thereof. For example, suppression of BTK can increase expression of IFN-7. For example, suppression of BTK can increase IL-2. For example, suppression of BTK can increase expression of IL-12.

Suppression of BTK can decrease the expression of one or more Th2 cell related markers. Suppression of BTK can decrease the expression of one or more Th2 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where BTK is not suppressed. The one or more Th2 related markers can include CCR3, CCR4, CCR7, CCR8, CD4, CD30, CD81, CD184, CD278, c-maf, CRTH2, Gata-3, GM-CSF, IFN yR, IgD, IL-1R, IL-4, IL-5, IL-6, IL-9, IL-10, IL-13, IL-15, ST2L/T1, Tim-1, or any combination thereof. In particular, the one or more Th2 related markers can include IL-4, IL-5, IL-6, IL-10, IL-13, IL-15 or any combination thereof. For example, suppression of BTK can decrease IL-4 expression. For example, suppression of BTK can decrease IL-5 expression. For example, suppression of BTK can decrease IL-6 expression. For example, suppression of BTK can decrease IL-10 expression. For example, suppression of BTK can decrease IL-13 expression. For example, suppression of BTK can decrease IL-15 expression.

Suppression of BTK can increase the ratio of Th1 T cells to Th2 T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of BTK can decrease the ratio of Th2 T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Cytokine release syndrome is a known complication of the treatment of hematologic malignancies with chimeric antigen receptor-modified (CAR) T cells or with T cell replete, HLA-haploidentical blood or marrow transplantation. In embodiments, inhibition of BTK can attenuate cytokine release syndrome after non-engrafting, CD8-depleted lymphocyte infusion.

Cytokine release syndrome is graded on a scale from 0 to 5. Suppression of BTK can decrease the cytokine release syndrome score to 0, 1, 2, 3, or 4.

Suppression of BTK can decrease the expression of IL-10 by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where BTK is not suppressed.

Suppression of BTK can decrease the percentage of pyroptotic leukocytes among total leukocytes by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where BTK is not suppressed.

The activity of BTK, as measured for example by phosphorylation of one of its substrates (such as 1-Phosphatidylinositol-4,5-bisphosphate phosphodiesterase gamma-2; PLC-γ2) can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

ii. ITK

Interleukin-2 (IL-2) inducible T cell kinase (ITK) is a non-receptor tyrosine kinase highly expressed in T cell lineages and regulates multiple aspects of T cell development and function, mainly through its function downstream of the T cell receptor.

ITK can be suppressed (i.e., inhibited or attenuated) with a pharmacological agent or by genetic modification. The pharmacological agent could be an inhibitor of ITK. Suitable inhibitors of ITK include, but are not limited to, aminothiazole, aminobenzimidazole, indole, pyridine or prn694. The inhibitor can be a small molecule, a small interfering RNA (siRNA), or short hairpin RNA (shRNA). The ITK inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

ITK can be suppressed by knocking out the ITK gene from the genome. Techniques for knocking out genes are known by those skilled in the art. Gene knock-out methods in the art include, but are not limited to, gene silencing, conditional knockout, homologous recombination, gene editing, knockout by mutation. Gene silencing can be achieved using, for example, RNA interference, siRNA or shRNA. Conditional knockout methods can be used to inactivate the ITK gene. A loss of function mutation can help to suppress gene function by creating a mutation in the ITK gene. Gene editing techniques that can be employed to suppress ITK include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress ITK. A mutation can be made in one or more of the protein domains of ITK.

Suppression of ITK can decrease the number of Th2-polarized T cells in the leukocytes. Suppression of ITK can increase the number of Th1-polarized T cells in the leukocytes. Suppression of ITK can promote differentiation of T cells to Th1. Suppression of ITK can decrease IL-10, IL-4, or IL-13 expression in the leukocytes. Suppression of ITK can increase IFN-7 expression in the leukocytes. Suppression of ITK can increase IL-12 expression in the leukocytes.

Suppression of ITK can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where ITK is not suppressed. Suppression of ITK can decrease the population of Th2 by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where ITK is not suppressed.

Suppression of ITK can increase the expression of one or more Th1 cell related markers. Suppression of ITK can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where ITK is not suppressed. The one or more Th1 related markers can include CCR1, CD4, CD26, CD94, CD1 19, CD183, CD195, CD212, GM-CSF, Granzyme B, IFN-α, IFN-7, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-7, IL-2, IL-12 or any combination thereof. For example, suppression of ITK can increase expression of IFN-7. For example, suppression of ITK can increase IL-2. For example, suppression of ITK can increase expression of IL-12.

Suppression of ITK can decrease the expression of one or more Th2 cell related markers. Suppression of ITK can decrease the expression of one or more Th2 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where ITK is not suppressed. The one or more Th2 related markers can include CCR3, CCR4, CCR7, CCR8, CD4, CD30, CD81, CD184, CD278, c-maf, CRTH2, Gata-3, GM-CSF, IFN yR, IgD, IL-1R, IL-4, IL-5, IL-6, IL-9, IL-10, IL-13, IL-15, ST2L/T1, Tim-1, or any combination thereof. In particular, the one or more Th2 related markers can include IL-4, IL-6, IL-10, IL-13, IL-15 or any combination thereof. For example, suppression of ITK can decrease IL-4 expression. For example, suppression of ITK can decrease IL-5 expression. For example, suppression of ITK can decrease IL-6 expression. For example, suppression of ITK can decrease IL-10 expression. For example, suppression of ITK can decrease IL-13 expression. For example, suppression of ITK can decrease IL-15 expression.

Suppression of ITK can increase the ratio of Th1 T cells to Th2 T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of ITK can decrease the ratio of Th2 T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

The activity of ITK can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

iii. PI3Kδ

PI3Kδ can be suppressed (i.e., inhibited or attenuated) with a pharmacological agent or by genetic modification. The pharmacological agent could be an inhibitor of PI3Kδ. Suitable inhibitors of PI3Kδ include, but are not limited to, idelalisib, copanlisib, duvelisib, umbralisib, ME-4401, RP6503, perifosine, buparlisib, or dactolisib. The inhibitor can be a small molecule, a small interfering RNA (siRNA), or short hairpin RNA (shRNA). The PI3Kδ inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

PI3Kδ can be suppressed by knocking out the PI3Kδ gene from the genome. Techniques for knocking out genes are known by those skilled in the art. Known gene knock-out methods in the art, include but are not limited to, gene silencing, conditional knockout, homologous recombination, gene editing, and knockout by mutation. Gene silencing can be achieved using, for example, RNA interference, siRNA or shRNA. For example, the RNA interference, siRNA or shRNA can be against CD25, foxp3, or Ezh2. Conditional knockout methods can be used to inactivate the PI3Kδ gene. A loss of function mutation can help to suppress gene function by creating a mutation in the PI3Kδ gene. Gene editing techniques that can be employed to suppress PI3Kδ include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress PI3Kδ. Alternatively, a mutation can be made in one or more of the protein domains.

Genetic modification of PI3Kδ can comprise deleting the gene for CD25, foxp3, or Ezh2.

Suppression of PI3Kδ can decrease the number or function of CD4+CD25+ foxp3+ regulatory T cells (T_{regs}) in the leukocytes. Suppression of PI3Kδ can increase the number of Tₕ1 polarized T cells in the leukocytes. Suppression of PI3Kδ can promote differentiation of T cells to Tₕ1. Suppression of PI3Kδ can decrease expression of TGFβ or IL-10 in the leukocytes. Suppression of PI3Kδ can increase IFN-7 expression in the leukocytes. Suppression of PI3Kδ can increase IL-12 expression in the leukocytes.

Suppression of PI3Kδ can increase the population of Tₕ1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where PI3Kδ is not suppressed. Suppression of PI3Kδ can decrease the population of Treg cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where PI3Kδ is not suppressed.

Suppression of PI3Kδ can increase the expression of one or more Tₕ1 cell related markers. Suppression of PI3Kδ can increase the expression of one or more Tₕ1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where PI3Kδ is not suppressed. The one or more Tₕ1 related markers can include CCR1, CD4, CD26, CD94, CD1 19, CD183, CD195, CD212, GM-CSF, Granzyme B, IFN-α, IFN-7, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Tₕ1 related markers can include IFN-7, IL-2, IL-12 or any combination thereof. For example, suppression of PI3Kδ can increase expression of IFN-7. For example, suppression of PI3Kδ can increase IL-2. For example, suppression of PI3Kδ can increase expression of IL-12.

Suppression of PI3Kδ can decrease the expression of one or more Treg cell related markers. Suppression of PI3Kδ can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Treg is not suppressed. The one or more T_{reg} related markers can include, TGFβ or IL-10 or any combination thereof. For example, suppression of PI3Kδ can decrease TGFβ expression. For example, suppression of PI3Kδ can decrease IL-10 expression.

Suppression of PI3Kδ can increase the ratio of Tₕ T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of PI3Kδ can decrease the ratio of Treg T cells to Tₕ1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

The activity of PI3Kδ can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

iv. GATA3

GATA3 can be suppressed (i.e., inhibited or attenuated) with a pharmacological agent or by genetic modification. The pharmacological agent could be an inhibitor of GATA3. The inhibitor can be a small molecule, a small interfering RNA (siRNA), or short hairpin RNA (shRNA). The GATA3 inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

GATA3 can be suppressed by knocking out the GATA3 gene from the genome. Techniques for knocking out genes are known by those skilled in the art. Gene knock-out methods in the art, include but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the GATA3 gene. A loss of function mutation can help to suppress gene function by creating a mutation in the GATA3 gene. Gene editing techniques that can be employed to suppress GATA3 can include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress GATA3. A mutation can be made one or more of the protein domains.

Suppression of GATA3 can increase the number of Tₕ1 polarized T cells in the leukocytes. Suppression of GATA3 can promote differentiation of T cells to Tₕ1. Suppression of GATA3 can decrease IL-10, IL-4, or IL-13 expression in the leukocytes. Suppression of GATA3 can increase IFN-7 expression in the leukocytes. Suppression of GATA3 can increase IL-12 expression in the leukocytes. Suppression of GATA3 can decrease the number of Tₕ2 polarized T cells in the leukocytes.

Suppression of GATA3 can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where GATA3 is not suppressed. Suppression of GATA3 can decrease the population of Th2 by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where GATA3 is not suppressed.

Suppression of GATA3 can increase the expression of one or more related Th1 cell related markers. Suppression of GATA3 can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where GATA3 is not suppressed. The one or more Th1 related markers can include CCR1, CD4, CD26, CD94, CD1 19, CD183, CD195, CD212, GM-CSF, Granzyme B, IFN-α, IFN-7, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-7, IL-2, IL-12 or any combination thereof. For example, suppression of GATA3 can increase expression of IFN-7. For example, suppression of GATA3 can increase IL-2. For example, suppression of GATA3 can increase expression of IL-12.

Suppression of GATA3 can decrease the expression of one or more related Th2 cell related markers. Suppression of GATA3 can decrease the expression of one or more Th2 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where GATA3 is not suppressed. The one or more Th2 related markers can include CCR3, CCR4, CCR7, CCR8, CD4, CD30, CD81, CD184, CD278, c-maf, CRTH2, Gata-3, GM-CSF, IFN yR, IgD, IL-1R, IL-4, IL-5, IL-6, IL-9, IL-10, IL-13, IL-15, ST2L/T1, Tim-1, or any combination thereof. In particular, the one or more Th2 related markers can include IL-4, IL-5, IL-6, IL-10, IL-13, IL-15 or any combination thereof. For example, suppression of GATA3 can decease IL-4 expression. For example, suppression of GATA3 can decrease IL-5 expression. For example, suppression of GATA3 can decrease IL-6 expression. For example, suppression of GATA3 can decrease IL-10 expression. For example, suppression of GATA3 can decrease IL-13 expression. For example, suppression of GATA3 can decrease IL-15 expression.

Suppression of GATA3 can increase the ratio of Th1 T cells to Th2 T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of GATA3 can decrease the ratio of Th2 T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Cytokine release syndrome is a known complication of the treatment of hematologic malignancies with chimeric antigen receptor-modified (CAR) T cells or with T cell replete, HLA-haploidentical blood or marrow transplantation. In embodiments, inhibition of GATA3 can attenuate cytokine release syndrome after non-engrafting, CD8-depleted donor lymphocyte infusion. Suppression of GATA3 can decrease cytokine release syndrome by about 5%, about 10%, about 15%, about 20%, about 25%, about 30%, about 35%, about 40%, about 45%, about 50%, about 60%, about 65%, about 70%, about 75%, about 80%, about 85%, about 90%, about 95% or greater relative to activity without suppression of GATA3.

Suppression of GATA3 can decrease the expression of IL-10 by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where GATA3 is not suppressed.

Suppression of GATA3 can decrease the expression of pyroptosis by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where GATA3 is not suppressed.

The activity of GATA3 can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

v. STAT3

STAT3 can be suppressed (i.e., inhibited) with a pharmacological agent or by genetic modification. The pharmacological agent could be an inhibitor of STAT3. The inhibitor can be a small molecule, a small interfering RNA (siRNA), or short hairpin RNA (shRNA). The STAT3 inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

STAT3 can be suppressed by knocking out the STAT3 gene from the genome. Techniques for knocking out genes are known by those skilled in the art. Gene knock-out methods in the art, include but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the STAT3 gene. A loss of function mutation can help to suppress gene function by creating a mutation in the STAT3 gene. Gene editing techniques that can be employed to suppress STAT3 can include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress STAT3. A mutation can be made one or more of the protein domains.

Suppression of STAT3 can increase the number of Th1 polarized T cells in the leukocytes. Suppression of STAT3 can promote differentiation of T cells to Th1. Suppression of STAT3 can decrease IL-10, IL-4, or IL-13 expression in the leukocytes. Suppression of STAT3 can increase IFN-7 expression in the leukocytes. Suppression of STAT3 can increase IL-12 expression in the leukocytes. Suppression of STAT3 can decrease the number of Th17 polarized T cells or Tfh polarized T cells in the leukocytes.

Suppression of STAT3 can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where STAT3 is not suppressed. Suppression of STAT3 can decrease the population of Th17 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where STAT3 is not suppressed. Suppression of STAT3 can decrease the population of Tfh cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where STAT3 is not suppressed.

Suppression of STAT3 can increase the expression of one or more related Th1 cell related markers. Suppression of STAT3 can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where STAT3 is not suppressed. The one or more Th1 related markers can include CCR1, CD4, CD26, CD94, CD1 19, CD183, CD195, CD212, GM-CSF, Granzyme B, IFN-α, IFN-7, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-7, IL-2, IL-12 or any combination thereof. For example, suppression of STAT3 can increase expression of IFN-7. For example, suppression of STAT3 can increase IL-2. For example, suppression of STAT3 can increase expression of IL-12.

Suppression of STAT3 can decrease the expression of one or more Treg cell related markers. Suppression of STAT3 can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Treg is not suppressed. The one or more Treg related markers can include, TGFβ or IL-10 or any combination thereof. For example, suppression of STAT3 can decrease TGFβ expression. For example, suppression of STAT3 can decrease IL-10 expression.

Suppression of STAT3 can decrease the expression of one or more Tfh cell related markers. Suppression of STAT3 can decrease the expression of one or more Tfh cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Tfh is not suppressed. The one or more Tfh related markers can include, IL-21, IL-4, or any combination thereof. For example, suppression of STAT3 can decrease IL-21 expression. For example, suppression of STAT3 can decrease IL-4 expression.

Suppression of STAT3 can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of STAT3 can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of STAT3 can increase the ratio of Th1 T cells to Tfh T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of STAT3 can decrease the ratio of Tfh T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

The activity of STAT3 can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

vi. Foxp3

Foxp3 can be suppressed (i.e., inhibited) with a pharmacological agent or by genetic modification. The pharmacological agent could be an inhibitor of foxp3. The inhibitor can be a small molecule, a small interfering RNA (siRNA), or short hairpin RNA (shRNA). The foxp3 inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

Foxp3 can be suppressed by knocking out the FOXP3 gene from the genome.

Techniques for knocking out genes are known by those skilled in the art. Gene knock-out methods in the art, include but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the FOXP3 gene. A loss of function mutation can help to suppress gene function by creating a mutation in the FOXP3 gene. Gene editing techniques that can be employed to suppress foxp3 can include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress foxp3. A mutation can be made one or more of the protein domains.

Suppression of foxp3 can increase the number of Th1 polarized T cells in the leukocytes. Suppression of foxp3 can promote differentiation of T cells to Th1. Suppression of foxp3 can decrease IL-10, IL-4, or IL-13 expression in the leukocytes. Suppression of foxp3 can increase IFN-7 expression in the leukocytes. Suppression of foxp3 can increase IL-12 expression in the leukocytes. Suppression of foxp3 can decrease the number of Treg polarized T cells in the leukocytes.

Suppression of foxp3 can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where foxp3 is not suppressed. Suppression of foxp3 can decrease the population of Treg cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where foxp3 is not suppressed.

Suppression of foxp3 can increase the expression of one or more related Th1 cell related markers. Suppression of foxp3 can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where foxp3 is not suppressed. The one or more Th1 related markers can include CCR1, CD4, CD26, CD94, CD1 19, CD183, CD195, CD212, GM-CSF, Granzyme B, IFN-α, IFN-7, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-7, IL-2, IL-12 or any combination thereof. For example, suppression of foxp3 can increase expression of IFN-7. For example, suppression of foxp3 can increase IL-2. For example, suppression of foxp3 can increase expression of IL-12.

Suppression of foxp3 can decrease the expression of one or more related Treg cell related markers. Suppression of foxp3 can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where foxp3 is not suppressed. The one or more Treg related markers can include, TGFβ or IL-10 or any combination thereof. For example, suppression of foxp3 can decrease TGFβ expression. For example, suppression of foxp3 can decrease IL-10 expression.

Suppression of foxp3 can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of foxp3 can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

The activity of foxp3 can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

vii. CD25

CD25 can be suppressed (i.e., inhibited) with a pharmacological agent or by genetic modification. The pharmacological agent could be an inhibitor of CD25. The inhibitor can be a small molecule, a small interfering RNA (siRNA), or short hairpin RNA (shRNA). The CD25 inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

CD25 can be suppressed by knocking out the CD25 gene from the genome. Techniques for knocking out genes are known by those skilled in the art. Gene knock-out methods in the art, include but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the CD25 gene. A loss of function mutation can help to suppress gene function by creating a mutation in the CD25 gene. Gene editing techniques that can be employed to suppress CD25 can include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress CD25. A mutation can be made one or more of the protein domains.

Suppression of CD25 can increase the number of Th1 polarized T cells in the leukocytes. Suppression of CD25 can promote differentiation of T cells to Tₕ1. Suppression of CD25 can decrease IL-10, IL-4, or IL-13 expression in the leukocytes. Suppression of CD25 can increase IFN-7 expression in the leukocytes. Suppression of CD25 can increase IL-12 expression in the leukocytes. Suppression of CD25 can decrease the number of Treg polarized T cells in the leukocytes.

Suppression of CD25 can increase the population of Tₕ1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where CD25 is not suppressed. Suppression of CD25 can decrease the population of Treg cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where CD25 is not suppressed.

Suppression of CD25 can increase the expression of one or more related Tₕ1 cell related markers. Suppression of CD25 can increase the expression of one or more Tₕ1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where CD25 is not suppressed. The one or more Tₕ1 related markers can include CCR1, CD4, CD26, CD94, CD1 19, CD183, CD195, CD212, GM-CSF, Granzyme B, IFN-α, IFN-7, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Tₕ1 related markers can include IFN-7, IL-2, IL-12 or any combination thereof. For example, suppression of CD25 can increase expression of IFN-7. For example, suppression of CD25 can increase IL-2. For example, suppression of CD25 can increase expression of IL-12.

Suppression of CD25 can decrease the expression of one or more related Treg cell related markers. Suppression of CD25 can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where CD25 is not suppressed. The one or more T_{reg} related markers can include, TGFβ or IL-10 or any combination thereof. For example, suppression of CD25 can decrease TGFβ expression. For example, suppression of CD25 can decrease IL-10 expression.

Suppression of CD25 can increase the ratio of Tₕ1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of CD25 can decrease the ratio of Treg T cells to Tₕ1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

The activity of CD25 can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

viii. Ezh2

Ezh2 can be suppressed (i.e., inhibited) with a pharmacological agent or by genetic modification. The pharmacological agent could be an inhibitor of Ezh2. The inhibitor can be a small molecule, a small interfering RNA (siRNA), or short hairpin RNA (shRNA). The Ezh2 inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

Ezh2 can be suppressed by knocking out the Ezh2 gene from the genome. Techniques for knocking out genes are known by those skilled in the art. Gene knock-out methods in the art, include but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the Ezh2 gene. A loss of function mutation can help to suppress gene function by creating a mutation in the Ezh2 gene. Gene editing techniques that can be employed to suppress Ezh2 can include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress Ezh2. A mutation can be made one or more of the protein domains.

Suppression of Ezh2 can increase the number of Tₕ1 polarized T cells in the leukocytes. Suppression of Ezh2 can promote differentiation of T cells to Tₕ1. Suppression of Ezh2 can decrease IL-10, IL-4, or IL-13 expression in the leukocytes. Suppression of Ezh2 can increase IFN-7 expression in the leukocytes. Suppression of Ezh2 can increase IL-12 expression in the leukocytes. Suppression of Ezh2 can decrease the number of Treg polarized T cells in the leukocytes.

Suppression of Ezh2 can increase the population of Tₕ1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Ezh2 is not suppressed. Suppression of Ezh2 can decrease the population of Treg cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Ezh2 is not suppressed.

Suppression of Ezh2 can increase the expression of one or more related Tₕ1 cell related markers. Suppression of Ezh2 can increase the expression of one or more Tₕ1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Ezh2 is not suppressed. The one or more Tₕ1 related markers can include CCR1, CD4, CD26, CD94, CD1 19, CD183, CD195, CD212, GM-CSF, Granzyme B, IFN-α, IFN-7, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Tₕ1 related markers can include IFN-7, IL-2, IL-12 or any combination thereof. For example, suppression of Ezh2 can increase expression of IFN-7. For example, suppression of Ezh2 can increase IL-2. For example, suppression of Ezh2 can increase expression of Ezh2.

Suppression of Ezh2 can decrease the expression of one or more related Treg cell related markers. Suppression of Ezh2 can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Ezh2 is not suppressed. The one or more Treg related markers can include, TGFβ or IL-10 or any combination thereof. For example, suppression of Ezh2 can decrease TGFβ expression. For example, suppression of Ezh2 can decrease IL-10 expression.

Suppression of Ezh2 can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of Ezh2 can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

The activity of Ezh2 can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

ix. Helios

Helios can be suppressed (i.e., inhibited) with a pharmacological agent or by genetic modification. The pharmacological agent could be an inhibitor of Helios. The inhibitor can be a small molecule, a small interfering RNA (siRNA), or short hairpin RNA (shRNA). The Helios inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

Helios can be suppressed by knocking out the IKZF2 gene from the genome. Techniques for knocking out genes are known by those skilled in the art. Gene knock-out methods in the art, include but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the IKZF2 gene. A loss of function mutation can help to suppress gene function by creating a mutation in the IKZF2 gene. Gene editing techniques that can be employed to suppress Helios include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress Helios. A mutation can be made one or more of the protein domains.

Suppression of Helios can increase the number of Tₕ1 polarized T cells in the leukocytes. Suppression of Helios can promote differentiation of T cells to Tₕ1. Suppression of Helios can decrease IL-10, IL-4, or IL-13 expression in the leukocytes. Suppression of Helios can increase IFN-7 expression in the leukocytes. Suppression of Helios can increase IL-12 expression in the leukocytes. Suppression of Helios can decrease the number of Treg polarized T cells in the leukocytes.

Suppression of Helios can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Helios is not suppressed. Suppression of Helios can decrease the population of Treg by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Helios is not suppressed.

Suppression of Helios can increase the expression of one or more related Th1 cell related markers. Suppression of Helios can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Helios is not suppressed. The one or more Th1 related markers can include CCR1, CD4, CD26, CD94, CD1 19, CD183, CD195, CD212, GM-CSF, Granzyme B, IFN-α, IFN-7, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-7, IL-2, IL-12 or any combination thereof. For example, suppression of Helios can increase expression of IFN-7. For example, suppression of Helios can increase IL-2. For example, suppression of Helios can increase expression of IL-12.

Suppression of Helios can decrease the expression of one or more related Treg cell related markers. Suppression of Helios can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Helios is not suppressed. The one or more Treg related markers can include, TGFβ or IL-10 or any combination thereof. For example, suppression of Helios can decrease TGFβ expression. For example, suppression of Helios can decrease IL-10 expression.

Suppression of Helios can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of Helios can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of Helios can attenuate exhaustion of CD8+ T cells. Suppression of Helios can decrease CD8+ T cell exhaustion by about 5%, about 10%, about 15%, about 20%, about 25%, about 30%, about 35%, about 40%, about 45%, about 50%, about 60%, about 65%, about 70%, about 75%, about 80%, about 85%, about 90%, about 95% or greater relative to activity without suppression of Helios.

Suppression of Helios can increase the population of CD8+ T cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Helois is not suppressed.

The activity of Helios can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

x. Blimpi

Blimpi can be suppressed (i.e., inhibited) with a pharmacological agent or by genetic modification. The pharmacological agent could be an inhibitor of Blimp1. The Blimp1 inhibitor can be a small molecule, a small interfering RNA (siRNA), or short hairpin RNA (shRNA). The Blimp1 inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

Blimp1 can be suppressed by knocking out the PRDM1 gene from the genome.

Techniques for knocking out genes are known by those skilled in the art. Gene knock-out methods in the art, include but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the PRDM1 gene. A loss of function mutation can help to suppress gene function by creating a mutation in the PRDM1 gene. Gene editing techniques that can be employed to suppress Blimpi include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress Blimp1. A mutation can be made one or more of the protein domains.

Suppression of Blimpi can increase the number of Th1 polarized T cells in the leukocytes. Suppression of Blimp1 can promote differentiation of T cells to Th1. Suppression of Blimpi can decrease IL-10, IL-4, or IL-13 expression in the leukocytes. Suppression of Blimp1 can increase IFN-7 expression in the leukocytes. Suppression of Blimp1 can increase IL-12 expression in the leukocytes. Suppression of Blimp1 can decrease the number of Treg polarized T cells in the leukocytes.

Suppression of Blimp1 can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Blimp1 is not suppressed. Suppression of Blimp1 can decrease the population of Treg by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Blimp1 is not suppressed.

Suppression of Blimp1 can increase the expression of one or more related Th1 cell related markers. Suppression of Blimp1 can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Blimp1 is not suppressed. The one or more Th1 related markers can include CCR1, CD4, CD26, CD94, CD1 19, CD183, CD195, CD212, GM-CSF, Granzyme B, IFN-α, IFN-7, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-7, IL-2, IL-12 or any combination thereof. For example, suppression of Blimp1 can increase expression of IFN-7. For example, suppression of Blimp1 can increase IL-2. For example, suppression of Blimp1 can increase expression of IL-12.

Suppression of Blimp1 can decrease the expression of one or more related Treg cell related markers. Suppression of Blimp1 can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Blimp1 is not suppressed. The one or more Treg related markers can include, TGFβ or IL-10 or any combination thereof. For example, suppression of Blimp1 can decrease TGFβ expression. For example, suppression of Blimp1 can decrease IL-10 expression.

Suppression of Blimp1 can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of Blimp1 can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of Blimp1 can attenuate exhaustion of CD8+ T cells. Suppression of Blimp1 can decrease CD8+ T cell exhaustion by about 5%, about 10%, about 15%, about 20%, about 25%, about 30%, about 35%, about 40%, about 45%, about 50%, about 60%, about 65%, about 70%, about 75%, about 80%, about 85%, about 90%, about 95% or greater relative to activity without suppression of Blimp1.

Suppression of Blimp1 can increase the population of CD8+ T cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Blimp is not suppressed.

The activity of Blimp1 can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

xi. TOX

TOX (thymocyte selection-associated HMG BOX) can be suppressed (i.e., inhibited) with a pharmacological agent or by genetic modification. The pharmacological agent could be an inhibitor of TOX. The inhibitor can be a small molecule, a small interfering RNA (siRNA), or short hairpin RNA (shRNA). The TOX inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

TOX can be suppressed by knocking out the TOX gene from the genome. Techniques for knocking out genes are known by those skilled in the art. Gene knock-out methods in the art, include but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the TOX gene. A loss of function mutation can help to suppress gene function by creating a mutation in the TOX gene. Gene editing techniques that can be employed to suppress TOX can include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress TOX. A mutation can be made one or more of the protein domains.

Suppression of TOX can attenuate exhaustion of CD8+ T cells. Suppression of TOX can decrease CD8+ T cell exhaustion by about 5%, about 10%, about 15%, about 20%, about 25%, about 30%, about 35%, about 40%, about 45%, about 50%, about 60%, about 65%, about 70%, about 75%, about 80%, about 85%, about 90%, about 95% or greater relative to activity without suppression of TOX.

Suppression of TOX can increase the population of CD8+ T cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TOX is not suppressed.

xii. IL-10

IL-10 can be suppressed (i.e., inhibited) with a pharmacological agent or by genetic modification. The pharmacological agent could be an inhibitor of IL-10. The inhibitor can be a small molecule, a small interfering RNA (siRNA), or short hairpin RNA (shRNA). The IL-10 inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

IL-10 can be suppressed by knocking out the IL-10 gene from the genome.

Techniques for knocking out genes are known by those skilled in the art. Gene knock-out methods in the art, include but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the IL-10 gene. A loss of function mutation can help to suppress gene function by creating a mutation in the IL-10 gene. Gene editing techniques that can be employed to suppress IL-10 can include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress IL-10. A mutation can be made one or more of the protein domains.

Suppression of IL-10 can decrease the number of Th2 polarized T cells in the leukocytes. Suppression of IL-10 can increase the number of Th1 polarized T cells in the leukocytes. Suppression of IL-10 can promote differentiation of T cells to Th1. Suppression of IL-10 can decrease IL-10, IL-4, or IL-13 expression in the leukocytes. Suppression of IL-10 can increase IFN-7 expression in the leukocytes. Suppression of IL-10 can increase IL-12 expression in the leukocytes.

Suppression of IL-10 can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed. Suppression of IL-10 can decrease the population of Th2 by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed.

Suppression of IL-10 can increase the expression of one or more related Th1 cell related markers. Suppression of IL-10 can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed. The one or more Th1 related markers can include CCR1, CD4, CD26, CD94, CD1 19, CD183, CD195, CD212, GM-CSF, Granzyme B, IFN-α, IFN-7, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-7, IL-2, IL-12 or any combination thereof. For example, suppression of IL-10 can increase expression of IFN-7. For example, suppression of IL-10 can increase IL-2. For example, suppression of IL-10 can increase expression of IL-12.

Suppression of IL-10 can decrease the expression of one or more related Th2 cell related markers. Suppression of IL-10 can decrease the expression of one or more Th2 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed. The one or more Th2 related markers can include CCR3, CCR4, CCR7, CCR8, CD4, CD30, CD81, CD184, CD278, c-maf, CRTH2, Gata-3, GM-CSF, IFN yR, IgD, IL-1R, IL-4, IL-5, IL-6, IL-9, IL-10, IL-13, IL-15, ST2L/T1, Tim-1, or any combination thereof. In particular, the one or more Th2 related markers can include IL-4, IL-6, IL-10, IL-13, IL-15 or any combination thereof. For example, suppression of IL-10 can decease IL-4 expression. For example, suppression of IL-10 can decrease IL-5 expression. For example, suppression of IL-10 can decrease IL-6 expression. For example, suppression of IL-10 can decrease IL-10 expression. For example, suppression of IL-10 can decrease IL-13 expression. For example, suppression of IL-10 can decrease IL-15 expression.

Suppression of IL-10 can increase the ratio of Th1 T cells to Th2 T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of IL-10 can decrease the ratio of Th2 T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

The activity of IL-10 can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

Suppression of IL-10 can encourage inflammation and production of pro-inflammatory cytokines in other T cells. Suppression of IL-10 can increase expression of IL-1, IL-12, IL-18, TNF-alpha, IFN-gamma, or GM-CSF.

Suppression of IL-10 can increase the expression of IL-1 by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed.

Suppression of IL-10 can increase the expression of IL-12 by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed.

Suppression of IL-10 can increase the expression of IL-18 by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed.

Suppression of IL-10 can increase the expression of TFN-alpha by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed.

Suppression of IL-10 can increase the expression of I by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed.

Suppression of IL-10 can increase the expression of IL-18 by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed.

Suppression of IL-10 can increase the expression of GM-CFS by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed.

xiii. SOCS1

SOCS1 can be suppressed (i.e., inhibited) with a pharmacological agent or by genetic modification. The pharmacological agent could be an inhibitor of SOCS1. The SOCS1 inhibitor can be a small molecule, a small interfering RNA (siRNA), or short hairpin RNA (shRNA). The SOCS1 inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

SOCS1 can be suppressed by knocking out the SOCS1 gene from the genome. Techniques for knocking out genes are known by those skilled in the art. Gene knock-out methods in the art include, but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the SOCS1 gene. A loss of function mutation can help to suppress gene function by creating a mutation in the SOCS1 gene. Gene editing techniques that can be employed to suppress SOCS1 include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress SOCS1. A mutation can be made one or more of the protein domains.

Suppression of SOCS1 can increase the number of Th1 polarized T cells in the leukocytes. Suppression of SOCS1 can promote differentiation of T cells to Th1. Suppression of SOCS1 can decrease IL-10, IL-4, or IL-13 expression in the leukocytes. Suppression of SOCS1 can increase IFN-7 expression in the leukocytes. Suppression of SOCS1 can increase IL-12 expression in the leukocytes. Suppression of SOCS1 can decrease the number of Treg polarized T cells in the leukocytes.

Suppression of SOCS1 can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where SOCS1 is not suppressed. Suppression of SOCS1 can decrease the population of Treg by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where SOCS1 is not suppressed.

Suppression of SOCS1 can increase the expression of one or more related Th1 cell related markers. Suppression of SOCS1 can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where SOCS1 is not suppressed. The one or more Th1 related markers can include CCR1, CD4, CD26, CD94, CD1 19, CD183, CD195, CD212, GM-CSF, Granzyme B, IFN-α, IFN-7, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-7, IL-2, IL-12 or any combination thereof. For example, suppression of SOCS1 can increase expression of IFN-7. For example, suppression of SOCS1 can increase IL-2. For example, suppression of SOCS1 can increase expression of IL-12.

Suppression of SOCS1 can decrease the expression of one or more related Treg cell related markers. Suppression of SOCS1 can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where SOCS1 is not suppressed. The one or more Treg related markers can include, TGFβ or IL-10 or any combination thereof. For example, suppression of SOCSI can decrease TGFβ expression. For example, suppression of SOCS1 can decrease IL-10 expression.

Suppression of SOCS1 can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of SOCS1 can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of SOCS1 can attenuate exhaustion of CD8+ T cells. Suppression of SOCS1 can decrease CD8+ T cell exhaustion by about 5%, about 10%, about 15%, about 20%, about 25%, about 30%, about 35%, about 40%, about 45%, about 50%, about 60%, about 65%, about 70%, about 75%, about 80%, about 85%, about 90%, about 95% or greater relative to activity without suppression of SOCS1.

Suppression of SOCS1 can increase the population of CD8+ T cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where SOCS1 is not suppressed.

The activity of SOCS1 can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

xiv. PD-1

PD-1 can be suppressed (i.e., inhibited) with a pharmacological agent or by genetic modification. The pharmacological agent could be an inhibitor of PD-1. The PD-1 inhibitor can be a small molecule, a small interfering RNA (siRNA), or short hairpin RNA (shRNA). The PD-1 inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

PD-1 can be suppressed by knocking out the PD-1 gene from the genome. Techniques for knocking out genes are known by those skilled in the art. Gene knock-out methods in the art include, but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the PD-1 gene. A loss of function mutation can help to suppress gene function by creating a mutation in the PD-1 gene. Gene editing techniques that can be employed to suppress PD-1 include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress PD-1. A mutation can be made one or more of the protein domains.

Suppression of PD-1 can increase the number of Th1 polarized T cells in the leukocytes. Suppression of PD-1 can promote differentiation of T cells to Th1. Suppression of PD-1 can decrease IL-10, IL-4, or IL-13 expression in the leukocytes. Suppression of PD-1 can increase IFN-7 expression in the leukocytes. Suppression of PD-1 can increase IL-12 expression in the leukocytes. Suppression of PD-1 can decrease the number of Treg polarized T cells in the leukocytes.

Suppression of PD-1 can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where PD-1 is not suppressed. Suppression of PD-1 can decrease the population of Treg by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where PD-1 is not suppressed.

Suppression of PD-1 can increase the expression of one or more related Th1 cell related markers. Suppression of PD-1 can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where PD-1 is not suppressed. The one or more Th1 related markers can include CCR1, CD4, CD26, CD94, CD1 19, CD183, CD195, CD212, GM-CSF, Granzyme B, IFN-α, IFN-7, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-7, IL-2, IL-12 or any combination thereof. For example, suppression of PD-1 can increase expression of IFN-7. For example, suppression of PD-1 can increase IL-2. For example, suppression of PD-1 can increase expression of IL-12.

Suppression of PD-1 can decrease the expression of one or more related Treg cell related markers. Suppression of PD-1 can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where PD-1 is not suppressed. The one or more Treg related markers can include, TGFβ or IL-10 or any combination thereof. For example, suppression of PD-1 can decrease TGFβ expression. For example, suppression of PD-1 can decrease IL-10 expression.

Suppression of PD-1 can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of PD-1 can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of PD-1 can attenuate exhaustion of CD8+ T cells. Suppression of PD-1 can decrease CD8+ T cell exhaustion by about 5%, about 10%, about 15%, about 20%, about 25%, about 30%, about 35%, about 40%, about 45%, about 50%, about 60%, about 65%, about 70%, about 75%, about 80%, about 85%, about 90%, about 95% or greater relative to activity without suppression of PD-1.

Suppression of PD-1 can increase the population of CD8+ T cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where PD-1 is not suppressed.

The activity of PD-1 can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

xv. LAG-3

LAG-3 can be suppressed (i.e., inhibited) with a pharmacological agent or by genetic modification. The pharmacological agent could be an inhibitor of LAG-3. The inhibitor can be a small molecule, a small interfering RNA (siRNA), or short hairpin RNA (shRNA). The LAG-3 inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

LAG-3 can be suppressed by knocking out the LAG-3 gene from the genome. Techniques for knocking out genes are known by those skilled in the art. Gene knock-out methods in the art, include but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the LAG-3 gene. A loss of function mutation can help to suppress gene function by creating a mutation in the LAG-3 gene. Gene editing techniques that can be employed to suppress LAG-3 can include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress LAG-3. A mutation can be made one or more of the protein domains.

Suppression of LAG-3 can increase the number of Th1 polarized T cells in the leukocytes. Suppression of LAG-3 can promote differentiation of T cells to Th1. Suppression of LAG-3 can decrease IL-10, IL-4, or IL-13 expression in the leukocytes. Suppression of LAG-3 can increase IFN-7 expression in the leukocytes. Suppression of LAG-3 can increase IL-12 expression in the leukocytes. Suppression of LAG-3 can decrease the number of Treg polarized T cells in the leukocytes.

Suppression of LAG-3 can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where LAG-3 is not suppressed. Suppression of LAG-3 can decrease the population of Treg cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where LAG-3 is not suppressed.

Suppression of LAG-3 can increase the expression of one or more related Th1 cell related markers. Suppression of LAG-3 can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where LAG-3 is not suppressed. The one or more Th1 related markers can include CCR1, CD4, CD26, CD94, CD1 19, CD183, CD195, CD212, GM-CSF, Granzyme B, IFN-α, IFN-7, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-7, IL-2, IL-12 or any combination thereof. For example, suppression of LAG-3 can increase expression of IFN-7. For example, suppression of LAG-3 can increase IL-2. For example, suppression of LAG-3 can increase expression of IL-12.

Suppression of LAG-3 can decrease the expression of one or more related Treg cell related markers. Suppression of LAG-3 can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where LAG-3 is not suppressed. The one or more Treg related markers can include, TGFβ or IL-10 or any combination thereof. For example, suppression of LAG-3 can decrease TGFβ expression. For example, suppression of LAG-3 can decrease IL-10 expression.

Suppression of LAG-3 can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of LAG-3 can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

The activity of LAG-3 can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

xvi. TNF-Alpha

TNF-alpha can be suppressed (i.e., inhibited) with a pharmacological agent or by genetic modification. The pharmacological agent could be an inhibitor of TNF-alpha. The TNF-alpha inhibitor can be a small molecule, a small interfering RNA (siRNA), or short hairpin RNA (shRNA). The TNF-alpha inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

TNF-alpha can be suppressed by knocking out the TNF-alpha gene from the genome. Techniques for knocking out genes are known by those skilled in the art. Gene knock-out methods in the art include, but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the TNF-alpha gene. A loss of function mutation can help to suppress gene function by creating a mutation in the TNF-alpha gene. Gene editing techniques that can be employed to suppress TNF-alpha include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress TNF-alpha. A mutation can be made one or more of the protein domains.

Suppression of TNF-alpha can increase the number of Th1 polarized T cells in the leukocytes. Suppression of TNF-alpha can promote differentiation of T cells to Th1. Suppression of TNF-alpha can decrease IL-10, IL-4, or IL-13 expression in the leukocytes. Suppression of TNF-alpha can increase IFN-γ expression in the leukocytes. Suppression of TNF-alpha can increase IL-12 expression in the leukocytes. Suppression of TNF-alpha can decrease the number of Treg polarized T cells in the leukocytes.

Suppression of TNF-alpha can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TNF-alpha is not suppressed. Suppression of TNF-alpha can decrease the population of Treg by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TNF-alpha is not suppressed.

Suppression of TNF-alpha can increase the expression of one or more related Th1 cell related markers. Suppression of TNF-alpha can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TNF-alpha is not suppressed. The one or more Th1 related markers can include CCR1, CD4, CD26, CD94, CD1 19, CD183, CD195, CD212, GM-CSF, Granzyme B, IFN-α, IFN-7, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-7, IL-2, IL-12 or any combination thereof. For example, suppression of TNF-alpha can increase expression of IFN-7. For example, suppression of TNF-alpha can increase IL-2. For example, suppression of TNF-alpha can increase expression of IL-12.

Suppression of TNF-alpha can decrease the expression of one or more related Treg cell related markers. Suppression of TNF-alpha can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TNF-alpha is not suppressed. The one or more Treg related markers can include, TGFβ or IL-10 or any combination thereof. For example, suppression of TNF-alpha can decrease TGFβ expression. For example, suppression of TNF-alpha can decrease IL-10 expression.

Suppression of TNF-alpha can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of TNF-alpha can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of TNF-alpha can attenuate exhaustion of CD8+ T cells. Suppression of TNF-alpha can decrease CD8+ T cell exhaustion by about 5%, about 10%, about 15%, about 20%, about 25%, about 30%, about 35%, about 40%, about 45%, about 50%, about 60%, about 65%, about 70%, about 75%, about 80%, about 85%, about 90%, about 95% or greater relative to activity without suppression of TNF-alpha.

Suppression of TNF-alpha can increase the population of CD8+ T cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TNF-alpha is not suppressed.

The activity of TNF-alpha can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

Cytokine release syndrome is a known complication of the treatment of hematologic malignancies with chimeric antigen receptor-modified (CAR) T cells or with T cell replete, HLA-haploidentical blood or marrow transplantation. In embodiments, inhibition of TNF-alpha can attenuate cytokine release syndrome after non-engrafting, CD8-depleted donor lymphocyte infusion.

Cytokine release syndrome is graded on a scale from 0 to 5. Suppression of TNF-alpha can decrease the cytokine release syndrome score to 0, 1, 2, 3, or 4.

Suppression of TNF-alpha can decrease the expression of IL-10 by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TNF-alpha is not suppressed.

Suppression of TNF-alpha can decrease the percentage of pyroptotic leukocytes among total leukocytes by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TNF-alpha is not suppressed.

The activity of TNF-alpha, as measured for example by phosphorylation of one of its substrates (such as 1-Phosphatidylinositol-4,5-bisphosphate phosphodiesterase gamma-2; PLC-γ2) can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

xvii. TGF-Beta Receptor II

TGF-beta Receptor II can be suppressed (i.e., inhibited) with a pharmacological agent or by genetic modification. The pharmacological agent could be an inhibitor of TGF-beta Receptor II. The TGF-beta Receptor II inhibitor can be a small molecule, a small interfering RNA (siRNA), or short hairpin RNA (shRNA). The TGF-beta Receptor II inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

TGF-beta Receptor II can be suppressed by knocking out the TGF-beta Receptor II gene from the genome. Techniques for knocking out genes are known by those skilled in the art. Gene knock-out methods in the art include, but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the TGF-beta Receptor II gene. A loss of function mutation can help to suppress gene function by creating a mutation in the TGF-beta Receptor II gene. Gene editing techniques that can be employed to suppress TGF-beta Receptor II include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress TGF-beta Receptor II. A mutation can be made one or more of the protein domains.

Suppression of TGF-beta Receptor II can increase the number of Th1 polarized T cells in the leukocytes. Suppression of TGF-beta Receptor II can promote differentiation of T cells to Th1. Suppression of TGF-beta Receptor II can decrease IL-10, IL-4, or IL-13 expression in the leukocytes. Suppression of TGF-beta Receptor II can increase IFN-7 expression in the leukocytes. Suppression of TGF-beta Receptor II can increase IL-12 expression in the leukocytes. Suppression of TGF-beta Receptor II can decrease the number of Treg polarized T cells in the leukocytes.

Suppression of TGF-beta Receptor II can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TGF-beta Receptor II is not suppressed. Suppression of TGF-beta Receptor II can decrease the population of Treg by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TGF-beta Receptor II is not suppressed.

Suppression of TGF-beta Receptor II can increase the expression of one or more related Th1 cell related markers. Suppression of TGF-beta Receptor II can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TGF-beta Receptor II is not suppressed. The one or more Th1 related markers can include CCR1, CD4, CD26, CD94, CD1 19, CD183, CD195, CD212, GM-CSF, Granzyme B, IFN-α, IFN-7, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-7, IL-2, IL-12 or any combination thereof. For example, suppression of TGF-beta Receptor II can increase expression of IFN-7. For example, suppression of TGF-beta Receptor II can increase IL-2. For example, suppression of TGF-beta Receptor II can increase expression of IL-12.

Suppression of TGF-beta Receptor II can decrease the expression of one or more related Treg cell related markers. Suppression of TGF-beta Receptor II can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TGF-beta Receptor II is not suppressed. The one or more Treg related markers can include, TGFβ or IL-10 or any combination thereof. For example, suppression of TGF-beta Receptor II can decrease TGFβ expression. For example, suppression of TGF-beta Receptor II can decrease IL-10 expression.

Suppression of TGF-beta Receptor II can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of TGF-beta Receptor II can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of TGF-beta Receptor II can attenuate exhaustion of CD8+ T cells. Suppression of TGF-beta Receptor II can decrease CD8+ T cell exhaustion by about 5%, about 10%, about 15%, about 20%, about 25%, about 30%, about 35%, about 40%, about 45%, about 50%, about 60%, about 65%, about 70%, about 75%, about 80%, about 85%, about 90%, about 95% or greater relative to activity without suppression of TGF-beta Receptor II.

Suppression of TGF-beta Receptor II can increase the population of CD8+ T cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TGF-beta Receptor II is not suppressed.

The activity of TGF-beta Receptor II can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

### B. Therapeutic Applications

The disclosure also relates to methods for treating a disease or condition, such as cancer. The method comprises administering to a subject in need thereof a lymphodepleting agent and/or an immune-stimulating agent and administering to the subject an allogenic lymphocyte composition as described herein.

The lymphodepleting agent can be a cytoreductive agent. Exemplary cytoreductive agents include, but are not limited to, an alkylating agent, alkyl sulphonates, nitrosoureas, triazene, antimetabolites, pyrimidine analog, purine analog, vinca alkaloids, epiodophyllotoxins, antibiotics, dirbromannitol, deoxyspergualine, dimethyl myleran and tiotepa.

The lymphodepleting agent can be a chemotherapeutic agent or a biologic agent. Exemplary chemotherapeutic agents and/or biologic agents include, but are not limited to, an antibody, a B cell receptor pathway inhibitor, a T cell receptor inhibitor, a PI3K inhibitor, an IAP inhibitor, an mTOR inhibitor, a radioimmunotherapeutic, a DNA damaging agent, a histone deacetylase inhibitor, a protein kinase inhibitor, a hedgehog inhibitor, an Hsp90 inhibitor, a telomerase inhibitor, a Jak1/2 inhibitor, a protease inhibitor, an IRAK inhibitor, a PKC inhibitor, a PARP inhibitor, a CYP3 A4 inhibitor, an AKT inhibitor, an Erk inhibitor, a proteosome inhibitor, an alkylating agent, an anti-metabolite, a plant alkaloid, a terpenoid, a cytotoxin, a topoisomerase inhibitor, a CD79A inhibitor, a CD79B inhibitor, a CD 19 inhibitor, a Lyn inhibitor, a Syk inhibitor, a PI3K inhibitor, a B1nk inhibitor, a PLCy inhibitor, a PKCP inhibitor, a CD22 inhibitor, a Bcl-2 inhibitor, an IRAK 1/4 inhibitor, a JAK inhibitor (e.g., ruxolitinib, baricitinib, CYT387, lestauritinib, pacritinib, TG101348, SAR302503, tofacitinib (Xeljanz), etanercept (Enbrel), GLPG0634, R256), a microtubule inhibitor, a Topo II inhibitor, anti-TWEAK antibody, anti-IL17 bispecific antibody, a CK2 inhibitor, anaplastic lymphoma kinase (ALK) and c-Met inhibitors, demethylase enzyme inhibitors such as demethylase, HDM, LSDI and KDM, fatty acid synthase inhibitors such as spirocyclic piperidine derivatives, glucocorticosteriod receptor agonist, fusion anti-CD 19-cytotoxic agent conjugate, antimetabolite, p70S6K inhibitor, immune modulators, AKT/PKB inhibitor, procaspase-3 activator PAC-1, BRAF inhibitor, lactate dehydrogenase A (LDH-A) inhibitor, CCR2 inhibitor, CXCR4 inhibitor, chemokine receptor antagonists, DNA double stranded break repair inhibitors, NOR202, GA-101, TLR2 inhibitor, Muromonab-CD3, rituximab (rituxan), carfilzomib, fludarabine, cyclophosphamide, vincristine, chlorambucil, ifosphamide, doxorubicin, mesalazine, thalidomide, revlimid, lenalidomide, temsirolimus, everolimus, fostamatinib, paclitaxel, docetaxel, ofatumumab, dexamethasone, bendamustine, CAL-101, ibritumomab, tositumomab, bortezomib, pentostatin, endostatin, ritonavir, ketoconazole, an anti-VEGF antibody, herceptin, cetuximab, cisplatin, carboplatin, docetaxel, erlotinib, etopiside, 5-fluorouracil, gemcitabine, ifosphamide, imatinib mesylate (Gleevec), gefitinib, erlotinib, procarbazine, irinotecan, leucovorin, mechlorethamine, methotrexate, oxaliplatin, paclitaxel, sorafenib, sunitinib, topotecan, vinblastine, GA-1101, dasatinib, Sipuleucel-T, disulfiram, epigallocatechin-3-gallate, salinosporamide A, ONX0912, CEP-18770, MLN9708, R-406, lenalinomide, spirocyclic piperidine derivatives, quinazoline carboxamide azetidine compounds, thiotepa, DWA2114R, NK121, IS 3 295, 254-5, alkyl sulfonates such as busulfan, improsulfan and piposulfan, aziridines such as benzodepa, carboquone, meturedepa and uredepa, ethylenimine, methylmelamines such as altretamine, triethylenemelamine, triethylenephosphoramide, triethylenethiophosphoramide and trimethylmelamine, chlornaphazine, estramustine, ifosfamide, mechlorethamine, oxide hydrochloride, novobiocin, phenesterine, prednimustine, trofosfamide, uracil mustard, nitrosoureas such as carmustine, chlorozotocin, fotemustine, lomustine, nimustine, ranimustine, antibiotics such as aclacinomycins, actinomycin, anthramycin, azaserine, bleomycins, cactinomycin, calicheamicin, carubicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin, antimetabolites such as methotrexate and 5-fluorouracil (5-FU), folic acid analogues such as denopterin, methotrexate, pteropterin, trimetrexate, purine analogs such as fludarabine, 6-mercaptopurine, thiamiprine, thioguanine, pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone, anti-adrenals such as aminoglutethimide, mitotane, trilostane, folic acid replenisher such as folinic acid, aceglatone, aldophosphamide glycoside, aminolevulinic acid, amsacrine, bestrabucil, bisantrene, edatrexate, defosfamide, demecolcine, diaziquone, eflornithine, elliptinium acetate, etoglucid, gallium nitrate, hydroxyurea, lentinan, lonidamine, mitoguazone, mitoxantrone, mopidamol, nitracrine, pentostatin, phenamet, pirarubicin, podophyllinic acid, 2-ethylhydrazide, procarbazine, polysaccharide-K, razoxane, sizofiran, spirogermanium, tenuazonic acid, triaziquone, 2, 2′,2″-trichlorotriethylamine, urethan, vindesine, dacarbazine, mannomustine, mitobronitol, mitolactol, pipobroman, gacytosine, cytosine arabinoside, taxoids, e.g., paclitaxel and docetaxel, 6-thioguanine, mercaptopurine, methotrexate, platinum analogs, platinum, etoposide (VP-16), ifosfamide, mitomycin C, mitoxantrone, vincristine, vinorelbine, Navelbine, Novantrone, teniposide, daunomycin, aminopterin, Xeloda, ibandronate, CPT1 1, topoisomerase inhibitor RFS 2000, difluoromethylornithine (DMFO), retinoic acid, esperamycins, capecitabine, and pharmaceutically acceptable salts, acids or derivatives of, anti-hormonal agents such as anti-estrogens including for example tamoxifen, raloxifene, aromatase inhibiting 4(5)-imidazoles, 4-hydroxytamoxifen, trioxifene, keoxifene, LY117018, onapristone and toremifene (Fareston), antiandrogens such as flutamide, nilutamide, bicalutamide, leuprolide and goserelin, ACK inhibitors such as AVL-263 (Avila Therapeutics/Celgene Corporation), AVL-292 (Avila Therapeutics/Celgene Corporation), AVL-291 (Avila Therapeutics/Celgene Corporation), BMS-488516 (Bristol-Myers Squibb), BMS-509744 (Bristol-Myers Squibb), CGI-1746 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) or a combination thereof.

The compositions and methods disclosed herein can used for any suitable cancer, including, but not limited to, bladder cancer, brain cancer, breast cancer, colorectal cancer, cervical cancer, gastrointestinal cancer, genitourinary cancer, head and neck cancer, lung cancer, ovarian cancer, prostate cancer, renal cancer, skin cancer, and testicular cancer, cardiac cancers, including, for example sarcoma, e.g., angiosarcoma, fibrosarcoma, rhabdomyosarcoma, and liposarcoma, myxoma, rhabdomyoma, fibroma, lipoma and teratoma, lung cancers, including, for example, bronchogenic carcinoma, e.g., squamous cell, undifferentiated small cell, undifferentiated large cell, and adenocarcinoma, alveolar and bronchiolar carcinoma, bronchial adenoma, sarcoma, lymphoma, chondromatous hamartoma, and mesothelioma, gastrointestinal cancer, including, for example, cancers of the esophagus, e.g., squamous cell carcinoma, adenocarcinoma, leiomyosarcoma, and lymphoma, cancers of the stomach, e.g., carcinoma, lymphoma, and leiomyosarcoma, cancers of the pancreas, e.g., ductal adenocarcinoma, insulinoma, glucagonoma, gastrinoma, carcinoid tumors, and vipoma, cancers of the small bowel, e.g., adenocarcinoma, lymphoma, carcinoid tumors, Kaposi's sarcoma, leiomyoma, hemangioma, lipoma, neurofibroma, and fibroma, cancers of the large bowel, e.g., adenocarcinoma, tubular adenoma, villous adenoma, hamartoma, and leiomyoma, genitourinary tract cancers, including, for example, cancers of the kidney, e.g., adenocarcinoma, Wilm's tumor (nephroblastoma), lymphoma, and leukemia, cancers of the bladder and urethra, e.g., squamous cell carcinoma, transitional cell carcinoma, and adenocarcinoma, cancers of the prostate, e.g., adenocarcinoma, and sarcoma, cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and limphoma, liver cancers, including, for example, hepatoma, e.g., hepatocellular carcinoma, cholangiocarcinoma, hepatoblastoma, angiosarcoma, hepatocellular adenoma, and hemangioma, bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors, nervous system cancers, including, for example, cancers of the skull, e.g., osteoma, hemangioma, granuloma, xanthoma, and osteitis defoinians, cancers of the meninges, e.g., meningioma, meningiosarcoma, and gliomatosis, cancers of the brain, e.g., astrocytoma, medulloblastoma, glioma, ependymoma, germinoma (pinealoma), glioblastoma multiform, oligodendroglioma, schwannoma, retinoblastoma, and congenital tumors, and cancers of the spinal cord, e.g., neurofibroma, meningioma, glioma, and sarcoma, gynecological cancers, including, for example, cancers of the uterus, e.g., endometrial carcinoma, cancers of the cervix, e.g., cervical carcinoma, and pre tumor cervical dysplasia, cancers of the ovaries, e.g., ovarian carcinoma, including serous cystadenocarcinoma, mucinous cystadenocarcinoma, unclassified carcinoma, granulosa thecal cell tumors, Sertoli Leydig cell tumors, dysgerminoma, and malignant teratoma, cancers of the vulva, e.g., squamous cell carcinoma, intraepithelial carcinoma, adenocarcinoma, fibrosarcoma, and melanoma, cancers of the vagina, e.g., clear cell carcinoma, squamous cell carcinoma, botryoid sarcoma, and embryonal rhabdomyosarcoma, and cancers of the fallopian tubes, e.g., carcinoma, hematologic cancers, including, for example, cancers of the blood, e.g., acute myeloid leukemia, chronic myeloid leukemia, acute lymphoblastic leukemia, chronic lymphocytic leukemia, myeloproliferative diseases, multiple myeloma, and myelodysplasia syndrome, Hodgkin's lymphoma, non-Hodgkin's lymphoma (malignant lymphoma) and Waldenstrom's macro globulinemia, skin cancers, including, for example, malignant melanoma, basal cell carcinoma, squamous cell carcinoma, Kaposi's sarcoma, moles dysplastic nevi, lipoma, angioma, dermatofibroma, keloids, psoriasis, and adrenal gland cancers, including, for example, neuroblastoma. In certain embodiments, when the disease is cancer, it may include a lung cancer tumor, a breast cancer tumor, a prostate cancer tumor, a brain cancer tumor, or a skin cancer tumor for example.

The subject can have a solid tumor. In some embodiments, the subject can have a sarcoma, carcinoma, or a neurofibromatoma. In some embodiments, the subject can have a colon cancer. In some embodiments, the subject can have a lung cancer. In some embodiments, the subject can have an ovarian cancer. In some embodiments, the subject can have a pancreatic cancer. In some embodiments, the subject can have a prostate cancer. In some embodiments, the subject can have a proximal or distal bile duct carcinoma. In some embodiments, the subject can have a breast cancer. In some embodiments, the subject can have a HER2-positive breast cancer. In some embodiments, the subject can have a HER2-negative breast cancer. In some embodiments, the subject has been treated for a solid tumor, and the method is applied to treat a subject as adjuvant therapy, that is the method is applied to the subject when the cancer is in a complete remission so as to prevent relapse of the cancer.

The subject can have a hematologic cancer. In some embodiments, the cancer is a leukemia, a lymphoma, a myeloma, a myelodysplastic syndrome, or a myeloproliferative neoplasm. In some embodiments, the cancer is a non-Hodgkin lymphoma. In some embodiments, the cancer is a Hodgkin lymphoma. In some embodiments, the cancer is a B-cell malignancy. In some embodiments, the B-cell malignancy is chronic lymphocytic leukemia (CLL), small lymphocytic lymphoma (SLL), diffuse large B-cell lymphoma (DLBCL), follicular lymphoma (FL), activated B-cell diffuse large B-cell lymphoma (ABC-DLBCL), germinal center diffuse large B-cell lymphoma (GCB DLBCL), primary mediastinal B-cell lymphoma (PMBL), Burkitt's lymphoma, immunoblastic large cell lymphoma, precursor B-lymphoblastic lymphoma, mantle cell lymphoma (MCL), B cell prolymphocytic leukemia, lymphoplasmacytic lymphoma, Waldenstrom macroglobulinemia, splenic marginal zone lymphoma, plasma cell myeloma, plasmacytoma, extranodal marginal zone B cell lymphoma, nodal marginal zone B cell lymphoma, mediastinal (thymic) large B cell lymphoma, intravascular large B cell lymphoma, primary effusion lymphoma, or lymphomatoid granulomatosis. In some embodiments, the cancer is a T cell malignancy. In some embodiments, the T cell malignancy is peripheral T cell lymphoma not otherwise specified (PTCL-NOS), anaplastic large cell lymphoma, angioimmunoblastic lymphoma, cutaneous T cell lymphoma, adult T cell leukemia/lymphoma (ATLL), blastic NK-cell lymphoma, enteropathy-type T cell lymphoma, hematosplenic gamma-delta T cell lymphoma, lymphoblastic lymphoma, nasal NK/T cell lymphomas, or treatment-related T cell lymphomas. In some embodiments, the subject can have multiple myeloma.

The subject can have a relapsed or refractory cancer.

The methods disclosed herein can further involve the administration of one or more additional agents to treat cancer, such as chemotherapeutic agents (e.g., Adriamycin, Cerubidine, Bleomycin, Alkeran, Velban, Oncovin, Fluorouracil, Thiotepa, Methotrexate, Bisantrene, Noantrone, Thiguanine, Cytaribine, Procarabizine), immuno-oncology agents (e.g., anti-PD-Li, anti-CTLA4, anti-PD-1, anti-CD47, anti-GD2), cellular therapies (e.g., CAR-T, T cell therapy, natural killer cell therapy, gamma delta T cell therapy), oncolytic viruses and the like.

Non-limiting examples of additional agents to treat cancer include acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, dactinomycin, daunorubicin hydrochloride, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, flurocitabine, fosquidone, fostriecin sodium, gemcitabine, gemcitabine hydrochloride, hydroxyurea, idarubicin hydrochloride, ifosfamide, ilmofosine, interleukin II (including recombinant interleukin II, or rIL2), interferon alpha-2a, interferon alpha-2b, interferon alpha-nl interferon alpha-n3, interferon beta-Ia, interferon gamma-Ib, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinzolidine sulfate, vinzolidine sulfate, vorozole, zeniplatin, zinostatin, zorubicin hydrochloride.

The methods disclosed herein can further comprise administration of an anti-tumor antibody/drug conjugate. The anti-tumor antibody/drug conjugate can include, but not limited to, rituximab, cetuximab, trastuzumab, and pertuzumab, brentuximab vedotin, gemtuzumab ozogamicin, trastuzumab emtansine, inotuzumab ozogamicin, glembatumumab vedotin, lorvotuzumab mertansine, cantuzumab mertansine, or milatuzumab-doxorubicin.

The methods disclosed herein can further comprise administering an antiviral agent. Exemplary anti-viral agents include, but are not limited to, acyclovir, famciclovir, ganciclovir, penciclovir, valacyclovir, valganciclovir, idoxuridine, trifluridine, brivudine, cidofovir, docosanol, fomivirsen, foscarnet, tromantadine, imiquimod, podophyllotoxin, entecavir, lamivudine, telbivudine, clevudine, adefovir, tenofovir, boceprevir, telaprevir, pleconaril, arbidol, amantadine, rimantadine, oseltamivir, zanamivir, peramivir, inosine, interferon (e.g., Interferon alfa-2b, Peginterferon alfa-2a), ribavirin/taribavirin, abacavir, emtricitabine, lamivudine, didanosine, zidovudine, apricitabine, stampidine, elvucitabine, racivir, amdoxovir, stavudine, zalcitabine, tenofovir, efavirenz, nevirapine, etravirine, rilpivirine, loviride, delavirdine, atazanavir, fosamprenavir, lopinavir, darunavir, nelfmavir, ritonavir, saquinavir, tipranavir, amprenavir, indinavir, enfuvirtide, maraviroc, vicriviroc, PRO 140, ibalizumab, raltegravir, elvitegravir, bevirimat, and vivecon.

The compositions disclosed herein are typically administered systemically, for example by intravenous injection or intravenous infusion. Other routes of administration can be used, such as orally, parenterally, intravenous, intravenously, intra-articularly, intraperitoneally, intramuscularly, subcutaneously, intracavity, transdermally, intrahepatically, intracranially, nebulization/inhalation, by installation via bronchoscopy, or intratumorally.

The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently. An “effective dose” refers to amounts of the active ingredient that are sufficient to affect the course and the severity of the disease, leading to the reduction or remission of such pathology and may be determined using known methods.

### C. Method of Preparing

The disclosure also relates to methods of preparing the allogeneic lymphocyte compositions disclosed herein. The method comprises obtaining a peripheral blood cell composition from a donor subject that is allogenic to a recipient subject or from a cell line or umbilical cord blood. The peripheral blood cell composition can be a whole blood product or an apheresis product. The peripheral blood cell composition can be obtained using means known in the art, for example through venipuncture. The peripheral blood cell composition comprises both CD8+ T cells and CD4+ T cells. The peripheral blood cell composition can be obtained from human or non-human subjects. Preferentially, the peripheral blood cell composition is obtained from a human.

The leukocytes from the donor subject can be mismatched to a recipient subject for at least one HLA Class II allele mismatch in the donor versus recipient (graft-versus-host) direction relative to the recipient subject. Alternatively, the donor can have at least one HLA class II allele mismatch relative to the recipient in the donor versus the recipient (graft-versus-host) direction and at least one HLA Class II allele match relative to the recipient. The HLA class II allele mismatch or match can be at HLA-DRB1, HLA-DQB1, or HLA-DPB1.

If the donor and recipient are ABO blood type incompatible and the allogenic leukocyte composition comprises a number of red blood cells, then making the allogenic lymphocyte composition can further comprise reducing the number of red blood cells. “ABO blood type incompatible” as used herein refers to when the recipient has a major ABO red blood cell incompatibility against the donor, e.g., the recipient is blood type O, and the donor is blood type A, B, or AB, the recipient is type A and the donor is type B or AB, or the recipient is type B and the donor is type A or AB. The number of red blood cells can comprise less than or equal to about 50 ml in packed volume. e.g., less than or equal to about 50 ml in packed volume, preferably less than or equal to about 30 ml in packed volume, further “packed volume” should be defined, for example, centrifugation of the lymphocyte composition would result is a packed volume of 50 ml or less of red blood cells, a measured volume sample of the lymphocyte composition could also be screened to provide a proportionally representative volume of packed blood cells.

Mononuclear cells are then isolated from the peripheral blood cell composition, for example by Ficoll-Hypaque gradient separation. Next, the number of CD8+ cells in the leukocytes can be depleted. The number of CD8+ cells in the leukocytes can be depleted by about 1 fold, about 2 fold, about 3 fold, about 4 fold, about 5 fold, about 6 fold, about 7 fold, about 8 fold, about 9 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 200 fold, about 300 fold, about 400 fold, about 500 fold, about 600 fold, about 700 fold, about 800 fold, about 900 fold, about 1,000 fold or greater relative to undepleted leukocytes.

The leukocytes are further modified to suppress BTK, ITK, or PI3Kδ, helios, blimp1, SOCS1, GATA3, IL-10, STAT3, TOX, CD25, foxp3, Ezh2, TGF-beta Receptor II, LAG-3, PD-1, TNF-alpha, or combinations thereof. Without wishing to be bound by theory or mechanism, the inventors believe that suppression of BTK, ITK, PI3Kδ, helios, blimp1, SOCS1, GATA3, IL-10, STAT3, TOX, CD25, foxp3, Ezh2, TGF-beta Receptor II, LAG-3, PD-1, TNF-alpha, or combinations thereof may promote naïve CD4+ T cells to differentiate to a state, such as type 1 (Th1) CD4+ T cells, that is favorable for helping effector cells of anti-tumor or anti-viral immunity, or prevent post-naïve CD4+ T cells from converting to cells with suboptimal helper activity for anti-tumor or anti-viral immunity. For example, a portion of the T cells may be preferentially differentiated to a CD4+ T cell sub-type, specifically Th1.

The method can comprise promoting differentiation of at least a portion of T cells toward Th1 CD4+ T cells. Suppression of BTK, ITK, PI3Kδ, helios, blimp1, SOCS1, GATA3, IL-10, STAT3, TOX, CD25, foxp3, Ezh2, TGF-beta Receptor II, LAG-3, PD-1, TNF-alpha, or combinations thereof can promote differentiation of a portion of T cells towards Th1 CD4+ T cells.

The method can comprise culturing the leukocytes in vitro.

The method of producing the allogeneic composition can further comprise stimulating antigen-specific lymphocytes in the composition with antigen-presenting cells pulsed with antigenic peptides.

The method of producing the allogeneic composition can further comprise adding one or more additional agents, such as a cytokine or antibodies.

The additional agent can be a cytokine. Exemplary cytokines that can be added include IL-2, IL-7, IL-12, IL-15, IL-18, IFNγ, IL-21, CCDCl34, GM-CSF, or LYG1.

The additional agent can be an antibody. Exemplary antibodies include an anti-IL3 antibody, an anti-IL-4 antibody, an anti-CD3 antibody, an anti-CD200 antibody or an anti-CD28 antibody.

The additional agent can be an inhibitor. Exemplary inhibitors include inhibitors of MEK 1/2, ERK, p38, Cox-2, Pi13k, c512, setdb1, or Got1.

Other exemplary agents include, but are not limited to, receptor agonists (e.g., RAR alpha or TLR), transcription factors (e.g., T-bet and Tbx21), lipoarabinomannans, or lipomannans derived from BCG cell bodies

### D. Definitions

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include plural forms unless the context clearly indicates otherwise. The terms “include,” “such as,” and the like are intended to convey inclusion without limitation, unless otherwise specifically indicated.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

The term “cancer” refers to the physiological condition in mammals in which a population of cells is characterized by uncontrolled proliferation, immortality, metastatic potential, rapid growth and proliferation rate and/or certain morphological features. Often cancers can be in the form of a tumor or mass, but may exist alone within the subject, or may circulate in the blood stream as independent cells, such a leukemic or lymphoma cells. The term cancer includes all types of cancers and metastases, including hematological malignancy, solid tumors, sarcomas, carcinomas and other solid and non-solid tumors. Examples of cancers include, but are not limited to, carcinoma, lymphoma, blastoma, sarcoma, and leukemia. More particular examples of such cancers include squamous cell cancer, small cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer (e.g., triple negative breast cancer), osteosarcoma, melanoma, colon cancer, colorectal cancer, endometrial (e.g., serous) or uterine cancer, salivary gland carcinoma, kidney cancer, liver cancer, prostate cancer, vulval cancer, thyroid cancer, hepatic carcinoma, and various types of head and neck cancers. Triple negative breast cancer refers to breast cancer that is negative for expression of the genes for estrogen receptor (ER), progesterone receptor (PR), and Her2/neu.

As used herein, the term “T cell exhaustion” refers to the progressive loss of effector function (loss of IL-2, TNF-α, and IFN-γ production, or failure to kill cells expressing the T cell's cognate antigen) and sustained expression of inhibitory receptors such as PD-1, T cell immunoglobulin domain, and mucin domain-containing protein 3 (Tim-3), CTLA-4, lymphocyte-activation gene 3 (LAG-3), and CD160 with a transcriptional program distinct from functional effector or memory T cells

The term “subject” herein to refers to any animal, such as any mammal, including but not limited to, humans, non-human primates, rodents, and the like. In some embodiments, the mammal is a mouse. In some embodiments, the mammal is a human.

As used herein, the term “therapeutically effective amount” refers to an amount of a compound described herein (i.e., a allogeneic lymphocyte composition) that is sufficient to achieve a desired pharmacological or physiological effect under the conditions of administration. For example, a “therapeutically effective amount” can be an amount that is sufficient to reduce the signs or symptoms of a disease or condition (e.g., a tumor). Those skilled in the art will appreciate that the therapeutic effects need not be complete or curative, as long as some benefit is provided to the subject. A therapeutically effective amount of a pharmaceutical composition can vary according to factors such as the disease state, age, sex, and weight of the individual, and the ability of the pharmaceutical composition to elicit a desired response in the individual. An ordinarily skilled clinician can determine appropriate amounts to administer to achieve the desired therapeutic benefit based on these and other considerations.

### EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compounds and methods in detail, the same will be more clearly understood by reference to the following examples, which are introduced for illustration only and not intended to be limiting.

### EXAMPLES

The present invention is further described by the following examples, which are not intended to be limiting in any way.

### Example 1. Validating Anti-Tumor Efficacy of Donor E7 Priming, Ex Vivo Expansion, and Th1 Polarization in a Mouse Model of E7-Expressing Head and Neck Cancer

The objective of experiment will be to verify the efficacy of exogenous CD4+ T cell help for endogenous anti-tumor effectors in a mouse model of HPV-associated head and neck cancer. Table 1 shows the agents and treatment protocol for the study.

TABLE 1
Agents and Treatment
Recipient = B6 × C3H (B6C3) F₁; Donor = BALB/c × C57BL/6 (CB6)
F₁ (haplo) or B6C3 F₁ (syngeneic)

| | | 50,000 | | | |
|---|---|---|---|---|---|
| | | mEER | CTX | | ICB |
| | | cells | 200 mg/kg | 20 million spleen cells IV d 15 | (day 14, |
| Group | No. | IV d 0 | IP day 14 | (or equivalent of cultured cells) | 17, 20) |
| 1 | 12 | + | − | − | − |
| 2 | 12 | + | + | − | − |
| 3 | 12 | + | + | Unprimed, unexpanded | − |
| | | | | CD8⁻ CB6 F₁ | |
| 4 | 12 | + | + | Unprimed, ex vivo expanded | − |
| | | | | CD8⁻ CB6 F₁ | |
| 5 | 12 | + | + | Primed, unexpanded | − |
| | | | | CD8⁻ CB6 F1 | |
| 6 | 12 | + | + | Primed, ex vivo expanded | − |
| | | | | CD8⁻ CB6 F1 | |
| 7 | 12 | + | + | Primed, unexpanded | + |
| | | | | CD8⁻ CB6 F1 | |
| 8 | 12 | + | + | Primed, ex vivo expanded | + |
| | | | | CD8⁻ CB6 F1 | |
| 9 | 12 | + | + | Primed, ITK , ex vivo | − |
| | | | | expanded, CD8⁻ CB6 F1 | |
| 10 | 12 | + | + | Primed, ITK , ex vivo | + |
| | | | | expanded, CD8⁻ CB6 F1 | |

CD8− refers to depleted CD8+ T cells. CD8+ depletion will be performed using a Miltenyi CD8 depletion column.

For ex vivo expansion, mouse dendritic cells (DCs) will be enriched using mouse pan dendritic cell isolation kit (Miltenyi Biotech). DCs will be pulsed with 1 μg/ml each of HPV16 E6 and E7 peptides (JPT Peptide Technologies) in RPMI 1640 containing 2 mmol/L L-Glutamine 100 U/ml penicillin, 100 μg/ml streptomycin and 10% heat inactivated fetal calf serum (complete media) for 2 hours. E6/E7 pulsed DCs will then be transferred into flasks containing mouse splenocytes (treated with 1 μm an ITK inhibitor for 1 hour prior to mixture) in a 1:20 ratio with complete media containing 25 U/ml of recombinant mouse IL-2 and 20 ng/ml recombinant mouse GMCSF and cultured for 5 days. Cells will then be washed and rested for two days in complete medium plus IL-2 and GM-CSF prior to restimulation.

ITK-samples (i.e., groups 9 and 10) will be exposed for 1 hour ex vivo to an ITK inhibitor at 1 μM prior to culturing with peptide pulsed dendritic cells plus cytokines.

ICB refers to immune checkpoint blockade. ICB will be performed using RMP1-14 (anti-PD1; BioXCell) 100 mg IP in 100 ml HBSS+10F.9G2 (anti-PD-L1; BioXCell) 100 mg IP in 100 ml HBSS on indicated days 2.

### Example 2. Characterizing the Fate and Efficacy of E7-Primed Donor T Cells that have been Expanded Ex Vivo in Conditions Designed to Favor the Th1 Phenotype of CD4+ T Cells

We previously showed that in a syngeneic cell transfer model, primed whole spleen was curative but primed, CD8-depleted spleen did not provide any benefit. We hypothesize that E7-primed CD4+ T cells were reprogrammed to alternate (i.e. non-Tₕ1) CD4+ phenotypes, such as Tₕ2 or T_{regs}, following infusion. Further, we hypothesize that the anti-tumor efficacy of the infusion in the syngeneic model will be augmented by expanding E7-specific T cells ex vivo in Tₕ1 conditions and by deleting ITK as a means of promoting retention of the Tₕ1 phenotype in vivo. By promoting the Tₕ1 phenotype among infused CD4+ T cells, we hypothesize that these manipulations will prevent conversion of the CD4+ T cells into T_{regs}.

The objective of this study will be to characterize the fate and efficacy of E7-primed donor T cells that have been expanded ex vivo in conditions designed to favor the Th1 phenotype of CD4+ T cells. Table 2 shows the agents and treatment protocol for the study.

TABLE 2
Agents and Treatment
Donor = B6.SJL; Recipient = C57BL/6

| | | 50,000 | Cy | |
|---|---|---|---|---|
| | | TC1 | 200 mg/kg | |
| Group | N | IV d 0 | IP d 14 | 20 million DLI d 15 |
| 1 | 19 | − | + | Primed→CD8⁻ spleen |
| 2 | 19 | + | + | − |
| 3 | 19 | + | + | Unprimed, whole spleen |
| 4 | 19 | + | + | Primed, whole spleen |
| 5 | 19 | + | + | Primed→CD8⁻ spleen |
| 6 | 19 | + | + | Primed→CD8⁻→ex vivo expanded |
| 7 | 19 | + | + | Primed→CD8⁻→Tₕ1 polarized |
| 8 | 19 | + | + | Primed→CD8⁻→Tₕ1 polarized→ITK |
| | | | | CRISPR |
| 9 | 19 | + | + | Primed→CD8⁻→Tₕ1 polarized→ITK |
| | | | | CRISPR + (dominant negative |
| | | | | TGFbRII or foxp3 KO) |

At one, two, and three weeks after the DLI, three mice per group will be sacrificed. Spleen cells will be stimulated with dendritic cells pulsed with overlapping pentadecapeptides of E7 (JPT Peptides). The state of the E7-specific donor CD4+ T cells will be characterized by intracellular staining for IFN-γ, IL-4, IL-17A, or foxp3, and extracellular staining for CD4 and CD45.1, followed by flow cytometry.

It is believed that the primed, E7-specific CD4 T cells are getting exhausted or turning into alternative CD4 phenotypes (e.g. Th2, Th17, Treg). In the syngeneic model, one can track the donor cells and at intervals after infusion some of the mice can be sacrificed and the E7-specific T cells tested for number and function.

The same experiment will be repeated using CB6 F1 donors and B6C3 F1 recipients.

### Example 3. Comparison of Different E7 Vaccines as Immunogens for Non-Engrafting Donor Lymphocyte Infusion

The objective of the experiment will be to determine the optimal vaccine for eliciting E6/E7-specific Th1 CD4+ T cells, and for augmenting the anti-tumor effect of CD8-depleted donor lymphocyte infusion.

First, the effect of one, two, or three administrations of vaccine on CD4+ T cell response to E6/E7 antigens of Human Papillomavirus type 16 (HPV16) will be characterized.

9 BALB/c×B6 mice per group will be vaccinated with one, two, or three weekly doses of either RNA-LPX mRNA vaccine, PDS0101, or PapiVax DNA vaccine. One week after each vaccination, three mice per group will be sacrificed. Either the splenocytes or vaccine-draining lymph node cells will be stimulated with either overlapping peptides of E6 of HPV16 (JPT peptides, Germany) or overlapping peptides of E7. Six hours after stimulation, extracellular CD4 and CD8 and intracellular interferon gamma (IFN-γ) and tumor necrosis factor alpha (TNF-α) will be stained and analyzed by flow cytometry. Table 3 shows the agents and treatment protocol for the study.

TABLE 3
Agents and Treatment

| | | One | Two | Three |
|---|---|---|---|---|
| Recipient | Vaccine | vaccine | vaccines | vaccines |
| BALB/c × C57BL/6 F₁ | RNA-LPX¹ | N = 3 | N = 3 | N = 3 |
| | (BioNTech) | | | |
| BALB/c × C57BL/6 F₁ | PDS0101² | N = 3 | N = 3 | N = 3 |
| | (PDS Biosciences) | | | |
| BALB/c × C57BL/6 F₁ | pcDNA3-CRT/E7³ | N = 3 | N = 3 | N = 3 |
| | (PapiVax) | | | |

¹RNA-LPX: 40 micrograms intravenously, weekly × 2 injections
²PDS0101: 100 microliters containing 300 micrograms R-DOTAP plus 40 micrograms HPV peptide mixture subcutaneously, weekly × 3 injections
³PapiVax vaccine (pcDNA3-CRT/E7): 25 micrograms IM, weekly × 3

Next, the anti-tumor effect of non-engrafting cell therapy will be characterized using donors vaccinated against either RNA-LPX, PDS0101, or PapiVax vaccine. Table 4 shows the agents and treatment protocol for the study.

TABLE 4
Agents and Treatment
Recipient = C57BL/6 × C3H (B6C3) F₁;
Donor = BALB/c × C57BL/6 (CB6) F₁

| | | 50,000 | | |
|---|---|---|---|---|
| | | TC1-Luc | Cytoxan | |
| | | cells | 200 mg/kg | |
| Group | N | IV d 0 | IP d 14 | 20 million CD8⁻ cells |
| 1 | 10 | + | + | − |
| 2 | 10 | + | + | RNA-LPX-vaccinated donor |
| 3 | 10 | + | + | PDS0101-vaccinated donor |
| 4 | 10 | + | + | pcDNA3-CRT/E7-vaccinated donor |

The mice will be imaged weekly and survival will be followed.

Once the optimal vaccine has been identified, we will determine whether recipient vaccination adds to donor vaccination. Table 5 shows the agents and treatment protocol for the study.

TABLE 5
Agents and Treatment
Donor = CB6 F₁; Recipient = B6C3 F₁

| | | 50,000 | Cy | 2 × 10⁷ | Recipient |
|---|---|---|---|---|---|
| | | TC1-Luc | 200 mg/kg | vaccinated, | vaccine, |
| Group | N | IV d 0 | IP d 14 | CD8- DLI d 15 | d 16, 23, 30 |
| 1 | 10 | + | + | − | − |
| 2 | 10 | + | + | − | + |
| 3 | 10 | + | + | + | − |
| 4 | 10 | + | + | + | + |

### Example 4. CRISPR-Mediated Ablation of Selected Genes in Donor CD4+ T Cells

The objective of the study is to test CRISPR-mediated ablation of selected genes in donor CD4+ T cells as a means of polarizing to the Tₕ1 pathway by preventing conversion to regulatory T cells in vivo and augmenting the anti-tumor effect of non-engrafting donor lymphocyte infusion. Table 6 shows the agents and treatment protocol for the study.

TABLE 6
Agents and Treatment
Recipient = C57BL/6; donor = BALB/c

| | | 10⁵ ID8-luci | Cy 200 mg/kg | 20 million CD8-depleted |
|---|---|---|---|---|
| Group | N | IP d 0 | IP d 14 | spleen cells IV |
| 1 | 10 | + | + | − |
| 2 | 10 | + | + | Untreated |
| 3 | 10 | + | + | BTK KO (CRISPR) |
| 4 | 10 | + | + | ITK KO (CRISPR) |
| 5 | 10 | + | + | BTK/ITK KO (CRISPR) |
| 6 | 10 | + | + | Foxp3 KO (CRISPR) |
| 7 | 10 | + | + | ITK/Foxp3 KO (CRISPR) |
| 8 | 10 | + | + | ITK/BTK/Foxp3 KO (CRISPR) |

For each of groups 2-7, BTK and ITK activity will be assayed following inhibition by measuring the amount of phospho-BTK or phospho-ITK protein following CD3/CD28 stimulation (see J. Dubovsky et al. Blood 122: 2539, 2013). The frequency of IL-4 versus IFN-gamma producing CD4+ T cells will be measured by intracellular cytokine staining and flow cytometry.

### Example 5. Compare In Vivo Priming Against E7, with or without Ex Vivo Restimulation, to Purely Ex Vivo Priming and Restimulation for Generating and Expanding E7-Specific CD4⁺ Th1 Cells and Augmenting Anti-Tumor Efficacy of Non-Engrafting Donor Lymphocyte Infusion

The objective of the study is to determine if donor vaccination can be eliminated from the protocol.

### A. Preparation of Donor Cells

CB6 F1 donor mice or splenocytes will be treated as shown below in Table 7.

TABLE 7
Agents and Treatment

| Group | Day-13 | Day-6 | Day 1 | Day 8 |
|---|---|---|---|---|
| A | In vivo prime | In vivo boost | In vivo boost | Ex vivo boost |
| B | − | In vivo prime | In vivo boost | In vivo boost |
| C | − | Ex vivo prime | Ex vivo boost | Ex vivo boost |
| D | − | − | Ex vivo prime | Ex vivo boost |
| E | | − | − | Ex vivo prime |

In vivo prime or boost will be with 25 μg pcDNA3-CRT/E7 intramuscularly

### B. Ex Vivo Priming or Boosting.

Mouse dendritic cells (DCs) will be enriched using a mouse pan dendritic cell isolation kit (Miltenyi Biotech). DCs will be pulsed with 1 μg/ml E7 peptide (JPT Peptide Technologies) in RPMI 1640 containing 2 mmol/L L-Glutamine 100 U/ml penicillin, 100 μg/ml streptomycin and 10% heat inactivated fetal calf serum (complete media) for 2 hours. E7 pulsed DCs will then be transferred into flasks containing mouse splenocytes (treated with 1 μm of an JTK inhibitor for 1 hour prior to mixture) in 1:20 ratio with complete media containing 25 U/ml of recombinant mouse IL-2 and 20 ng/ml recombinant mouse GMCSF and cultured for 5 days. Cells will then be washed and rested for two days in complete medium plus IL-2 and GM-CSF prior to restimulation. Table 8 shows the agents and treatment protocol for the study.

TABLE 8
Agents and Treatment
Recipient mice = B6 × C3H (B6C3) F₁

| | | 50,000 TC1 | Cy 200 mg/kg | 20 million |
|---|---|---|---|---|
| Group | N | IV d 0 | IP d 14 | CD8- spleen IV* d 15 |
| 1 | 10 | + | + | − |
| 2 | 10 | + | + | Unprimed |
| 3 | 10 | + | + | Group A |
| 4 | 10 | + | + | Group B |
| 5 | 10 | + | + | Group C |
| 6 | 10 | + | + | Group D |
| 7 | 10 | + | + | Group E |

*The dose of spleen cells in groups A, C-E is the equivalent of 20 million input cells. For example, if 500 million cells were initially put into culture in group C and on day 15 there are 50 million cells remaining, then each mouse gets 1/25^{th} of the remaining cells or 2 million cells each.

On day 15, the frequency of E7-specific Th1 cells among total donor cells from groups 2-7 will be determined by intracellular cytokine staining and flow cytometry. Mouse splenocytes will be stimulated with 1 μg/ml E7 peptide with or without 5 μg/ml Brefeldin A in complete media for 5 hours. Cells will then be washed and stained with IFN-gamma and TNF-alpha along with CD4 and CD8.

### Example 6. The Effect of ITK Deletion in Donor CD4⁺ T Cells, Alone or in Combination with BTK Deletion, on the Anti-Lymphoma Efficacy of CD8-Depleted Donor Lymphocyte Infusion

The objective of the study will be to test whether the donor strain and its bias toward Tₕ1 (e.g., C57BL/6) or Tₕ2 (e.g. BALB/c) affects anti-tumor immunity of CD8-depleted DLI and its augmentation by BTK or JTK deletion in donor CD4+ T cells. Table 9 shows the tumor types, strain of origin, and donor strains for the study.

TABLE 9
Tumor types and stains of origin and donor

| Tumor | Strain of origin | Donor strain |
|---|---|---|
| A20 | BALB/c | C57BL/6 |
| BL3750³ | C57BL/6 | BALB/c |
| EL4-Luc2 | C57BL/6 | BALB/c |

The design of the experiment will be the same for all three tumor types, with the only difference being the tumor dose and the donor and recipient strains. Table 10 shows the treatment protocol for the study.

TABLE 10
Treatment protocol

| | | | | | 20 million |
|---|---|---|---|---|---|
| | | Tumor | | Ex vivo | CD8- donor |
| | | cells IV | Cyclophosphamide | treatment × | spleen cells |
| Group | N | day 0* | 200 mg/kg IP d 14 | 1 hr d 15 | IV d 15 |
| 1 | 10 | + | + | − | − |
| 2 | 10 | + | + | − | + |
| 3 | 10 | + | + | ITK KO | + |
| 4 | 10 | + | + | BTK + ITK KO | + |

Dose of cells: A20, 1 million; BL3750, 100,000; EL4-Luc2, 50,000

### Example 7. Efficacy of Non-Engrafting Donor Lymphocyte Infusion Against Common Malignancies (Lung, Breast, Ovarian)

The objective of the study was to test whether non-engrafting DLI can integrate into the treatment of common malignancies and synergize with checkpoint blockade. Lung cancer, breast cancer, and ovarian cancer will be studied.

### A. Lung Cancer

Tables 11 and 12 show the treatment protocol for the study in lung cancer

TABLE 11
Treatment protocol
Recipient strain = C57BL/6; Donor (lymphocyte) strain = BALB/c

| | | LLC cells | CTX* IV | | |
|---|---|---|---|---|---|
| Group | N | IV d 0 | d 14 | CD8-DLI d 15 | ICI* d 14, 17, 20 |
| 1 | 10 | + | − | − | − |
| 2 | 10 | + | + | − | − |
| 3 | 10 | + | + | Untreated | − |
| 4 | 10 | + | + | ITK KO (CRISPR) | − |
| 5 | 10 | + | + | − | + |
| 6 | 10 | + | + | Untreated | + |
| 7 | 10 | + | − | ITK KO (CRISPR) | + |

*CTX = cyclophosphamide 200 mg/kg intraperitoneally; CD8-DLI = CD8-depleted donor lymphocyte infusion; ICI = immune checkpoint inhibitor (anti-PD-1 + anti-PD-L1)
Follow survival

TABLE 12
Treatment protocol
Recipient = B6 × C3H (B6C3) F₁; donor stain = BALB/c × B6 (CB6) F₁

| | | 50,000 TC1 | CTX* | | ICI* d 14, |
|---|---|---|---|---|---|
| Group | N | cells IV | IV d 14 | CD8- DLI d 15 | 17, 20 |
| 1 | 10 | + | + | − | − |
| 2 | 10 | + | + | Untreated | − |
| 3 | 10 | + | + | ITK KO (CRISPR) | − |
| 4 | 10 | + | + | E7-primed, ITK KO | − |
| 5 | 10 | + | + | − | + |
| 6 | 10 | + | + | Untreated | + |
| 7 | 10 | + | + | ITK KO (CRISPR) | + |
| 8 | 10 | + | + | E7-primed, ITK KO | + |

*ICI = immunologic checkpoint inhibitor RMP1-14 (anti-PD1; BioXCell) 100 mg IP in 100 ml HBSS + 10F.9G2 (anti-PD-L1; BioXCell) 100 mg IP in 100 ml HBSS on days 14, 17, 20

### B. Breast Cancer

Table 13 shows the treatment protocol for the study in breast cancer.

### C. Ovarian Cancer

TABLE 13
Treatment protocol
Recipient strain = BALB/c; donor strain = C57BL/6

| | | 104 4T1-luc | CTX* IV | CD8-DLI | ICI* d 14, |
|---|---|---|---|---|---|
| Group | N | cells IV d 0 | d 14 | d 15 | 17, 20 |
| 1 | 10 | + | − | − | − |
| 2 | 10 | + | + | − | − |
| 3 | 10 | + | + | Untreated | − |
| 4 | 10 | + | + | ITK KO | − |
| 5 | 10 | + | + | − | + |
| 6 | 10 | + | + | Untreated | + |
| 7 | 10 | + | + | ITK KO | + |

*ICI = immunologic checkpoint inhibitor RMP1-14 (anti-PD1; BioXCell) 100 mg IP in 100 ml HBSS + 10F.9G2 (anti-PD-L1; BioXCell) 100 mg IP in 100 ml HBSS on days 14, 17, 20

Table 14 shows the treatment protocol for the study in breast cancer.

TABLE 14
Treatment protocol
Recipient strain = C57BL/6; donor strain = BALB/c

| | | 10⁵ ID8-luci | CTX* IV | CD8- DLI | ICI* d 14, |
|---|---|---|---|---|---|
| Group | N | IP d 0 | d 14 | d 15 | 17, 20 |
| 1 | 10 | + | − | − | − |
| 2 | 10 | + | + | − | − |
| 3 | 10 | + | + | Untreated | − |
| 4 | 10 | + | + | ITK KO | − |
| | | | | (CRISPR) | |
| 5 | 10 | + | + | − | + |
| 6 | 10 | + | + | Untreated | + |
| 7 | 10 | + | + | ITK KO | + |
| | | | | (CRISPR) | |

*ICI = immunologic checkpoint inhibitor RMP1-14 (anti-PD1; BioXCell) 100 mg IP in 100 ml HBSS + 10F.9G2 (anti-PD-L1; BioXCell) 100 mg IP in 100 ml HBSS on days 14, 17, 20

### Example 8. The Effect of Donor Neoantigen Vaccination on the Anti-Tumor Potency of CD8-Depleted, Non-Engrafting DLI Against a Neoantigen-Expressing Tumor

The objective of the study is to test whether the donor vaccination strategy can be employed to treat sporadic tumors via vaccination against tumor neoantigens.

In this experiment we will test two methods to increase the frequency of neoAg-specific CD4+ T cells in the CD8-depleted NEDLI: 1) in vivo vaccination with a CD4+ T cell neoepitope, without or with subsequent neopeptide stimulation ex vivo; or 2) in vitro “priming” using serial stimulation of CD4+ T cells with neopeptide+ DCs. The design of the experiment is shown in the Table 15 below.

TABLE 15
Treatment protocol

| | | | | | | 2 × 10⁷ |
|---|---|---|---|---|---|---|
| | | | 10⁵ | Cy 200 | | CD8⁻ spl |
| | Recipient | | B16-F10 | mg/kg | Donor | cells IV |
| Group | strain | N | IV d 0 | IP d 14 | strain | d 15 |
| 1 | B6 x C3H F₁ | 14 | + | + | B6C3 F₁ | WT* primed |
| 2 | ″ | 14 | + | + | ″ | Mut* primed |
| 3 | ″ | 14 | + | + | ″ | Mut primed + ex |
| | | | | | | vivo cultured |
| 4 | ″ | 14 | + | + | ″ | Ex vivo culture |
| | | | | | | with M30 × 2 wks |
| 5 | ″ | 14 | + | + | CB6 F₁ | WT primed |
| 6 | ″ | 14 | + | + | ″ | Mut primed |
| 7 | ″ | 14 | + | + | ″ | Mut primed + ex |
| | | | | | | vivo cultured |
| 8 | ″ | 14 | + | + | ″ | Ex vivo culture |
| | | | | | | with M30 × 2 wks |

*WT = PSKPSFQEFVDWEKVSPELNSTDQPFL; Mut(M30) = PSKPSFQEFVDWENVSPELNSTDQPFL

The B16-F10 melanoma of C57BL/6 (B6; H-2b) origin grows in F1 hybrids; immunogenic CD4+ neo-epitopes have been identified (See reference 4). 1) B6×C3H (B6C3; H-2bxk) F1 or MHC-haploidentical BALB/c×B6 (CB6; H-2bxd) F1 mice will be vaccinated with either the mutant neo-epitope M30 (groups 2, 6), encoded from the Kinesin family member 18b gene (Kif18b), or with the corresponding wild type peptide (groups 1, 5), the vaccine comprising 100 μg synthetic peptide and 50 μg poly(I:C) injected into the lateral flank in a volume of 200 μl phosphate buffered saline. The efficacy of vaccination and the phenotype (CD4 vs CD8) of responding cells will be tested by flow cytometry and intracellular cytokine staining (ICS) for interferon gamma (IFN=gamma) or tumor necrosis factor alpha (TNF-alpha), as described in reference 5. Two weeks after vaccination, spleen cells of euthanized donor mice will be depleted of CD8+ cells and infused into B16-F10 bearing B6C3 mice treated with Cy the day before infusion.

Alternatively, CD8-depleted cells from immunized donors will be cultured for 5 days with M30-pulsed, donor DCs for five days prior to infusion (groups 3, 7); 2) Spleen cells from naïve B6C3 or CB6 F1 mice will be stimulated weekly×2 with M30-pulsed autologous dendritic cells plus 20 U/ml IL-2 (groups 4, 8). The frequency of M30-specific, IFNgamma+ CD4+ T cells will be measured by ICS before and after ex vivo stimulation. NeoAg-specific CD4+ T cells can be purified using the IFNgamma capture assay (Miltenyi Biotec) and expanded further using beads coated with anti-CD3 and anti-CD28.

### REFERENCES

1. Williams R, Lee D W, Elzey B D, Anderson M E, Hostager B S, Lee J H. Preclinical models of HPV+ and HPV− HNSCC in mice: an immune clearance of HPV+ HNSCC. Head & Neck: Journal for the Sciences and Specialties of the Head and Neck. 2009; 31(7):911-918.
2. Ahrends T, Babala N, Xiao Y, Yagita H, van Eenennaam H, Borst J. CD27 Agonism Plus PD-1 Blockade Recapitulates CD4+ T-cell Help in Therapeutic Anticancer Vaccination. Cancer Res. 2016; 76(10):2921-2931.
3. Minard-Colin V, Xiu Y, Poe J C, et al. Lymphoma depletion during CD20 immunotherapy in mice is mediated by macrophage FcγRI, FcγRIII, and FcγRIV. Blood. 2008; 112(4):1205-1213.
4. Castle J C, Kreiter S, Diekmann J, Lower M, Roemer N, Graaf J. Exploiting the mutanome for tumor vaccination. Cancer Res. 2012; 72.
5. Kreiter S, Vormehr M, van de Roemer N, et al. Mutant MHC class II epitopes drive therapeutic immune responses to cancer. Nature. 2015; 520(7549):692-696.

### Example 9. Depletion of CD8+ T Cells

CD8+ T cells will be depleted from human blood products using the CliniMACS system with the CliniMACS® CD8 reagent. CD8+ T cells will be labeled with a monoclonal antibody linked to super-paramagnetic particles and will be depleted from the blood product by passage through the CliniMACS system, which incorporates a strong permanent magnet and a separation column with a ferromagnetic matrix to remove the labeled cells.

### Example 10. Differential Effects of Single Gene Deletions on the Anti-Tumor Effects of CD4+ T Cells from MHC Haploidentical Donor Vaccinated Against a Tumor Antigen

Previous experiments established that prior vaccination of healthy donors against a tumor antigen (E7 of human papillomavirus type 16 [HPV16]) augmented the anti-tumor efficacy of donor CD8-depleted donor lymphocytes infused after cyclophosphamide treatment of MHC-haploidentical recipients with advanced, E7-expressing tumors. Although CD4+ T cells from vaccinated donors modestly prolonged survival of the tumor-bearing recipients, all recipients ultimately succumbed to progressive tumor. See, U.S., Publication No.: 2022/0163997.

It is hypothesized that growing tumors ultimately polarize infused CD4+ T cells to become ineffective at providing help for anti-tumor immunity and that deletion of key genes involved in CD4+ T cell differentiation/polarization could prevent loss of CD4+ T cell help, thereby sustaining an anti-tumor immune response.

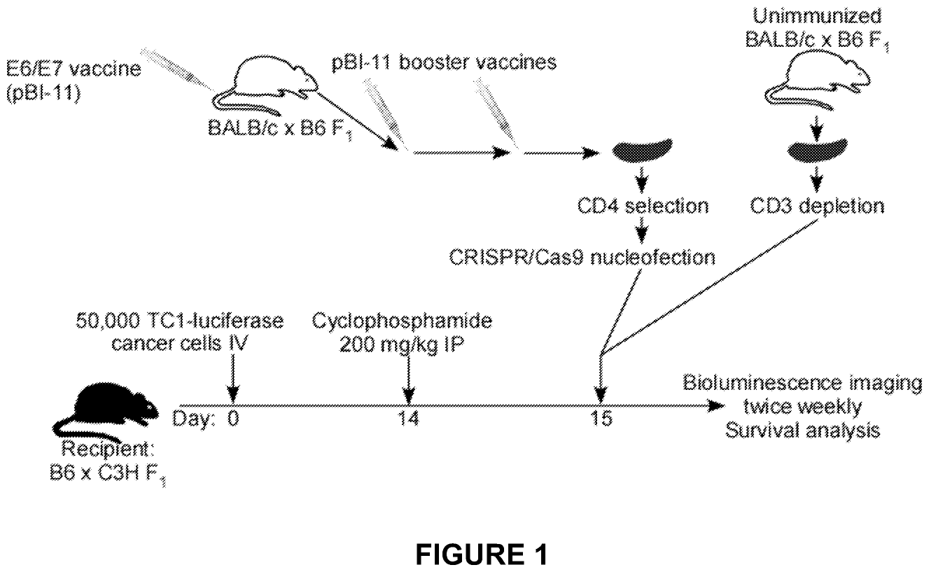

To test this hypothesis, BALB/c×C57BL/6 (CB6 F1) donors were vaccinated weekly for three doses with an intramuscular injection of 25 micrograms of pBI-11, a DNA vaccine encoding the E6 and E7 antigens of HPV16. One week after the third vaccine, CD4+ T cells were enriched to near purity using immunomagnetic beads and left untreated, transfected with a Cas9 nucleoprotein, or the Cas9 nucleoprotein plus guide RNAs to inactivate single genes: interleukin 2-inducible T cell kinase (ITK), forkhead box p3 (FOXP3), transforming growth factor-beta receptor type II (TGFBR2), suppressor of cytokine signaling-1 (SOCS1), or programmed death molecule-1 (PDCD1). After resting the cells overnight, 2.5 million unvaccinated, vaccinated but not nucleofected, vaccinated and Cas9 nucleofected, or single gene deficient CD4+ T cells were co-injected with 5 million syngeneic (CB6 F1), CD3-depleted spleen cells into MHC-haploidentical C57BL/6×C3H (B6C3 F1) mice that had received 50,000 TC-1 tumor cells (expressing E6 and E7 of HPV16) 14 days earlier and cyclophosphamide 200 mg/kg intraperitoneally the day before infusion. See, FIG. 1.

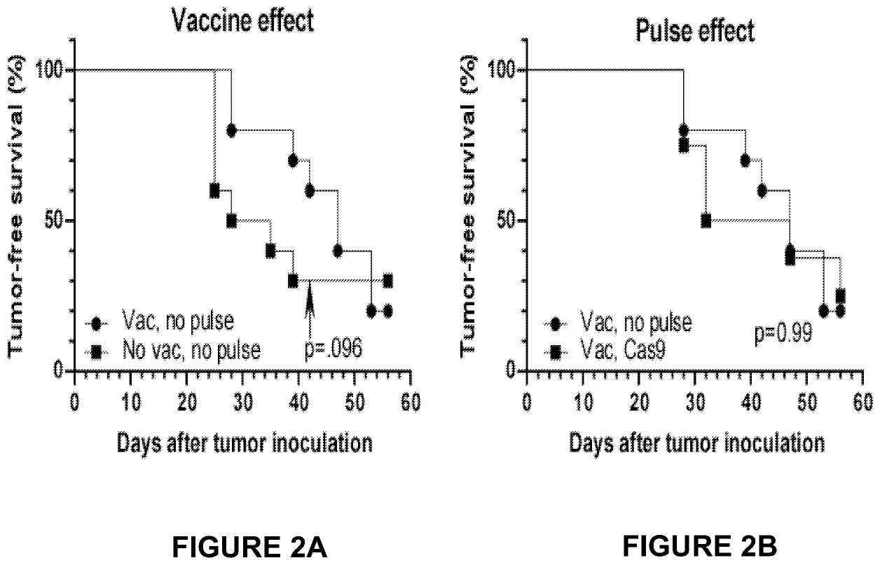

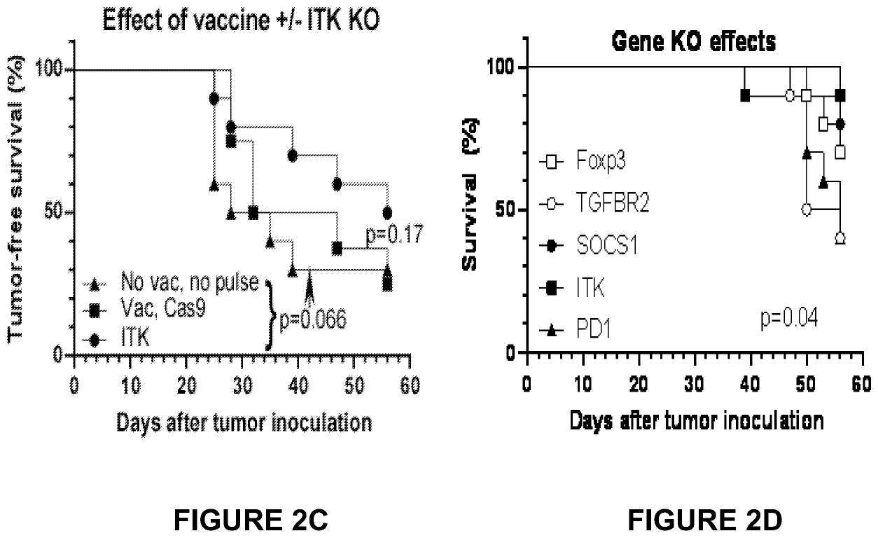

Donor vaccination against HPV E7 augmented the anti-tumor efficacy of cyclophosphamide CD8-depleted, MHC-haploidentical donor lymphocyte infusion, with difference in tumor-free survival of TC1-luci bearing recipients of unvaccinated versus vaccinated donor cells approaching statistical significance by day 42 after tumor inoculation (p=0.096; FIG. 2A). Nucleofection with the Cas9 reagent but without gene knockout had no significant effect on tumor-free survival of recipients of donor lymphocyte infusion (FIG. 2B; p=0.99). In contrast, CRISPR-mediated deletion of the gene for ITK in donor CD4+ T cells further augmented tumor-free survival compared to recipients of vaccinated CD4+ T cells without ITK deletion (p=0.17) or to recipients of CD4+ T cells from unvaccinated donors (p˜0.07; FIG. 2C). Deletions of distinct single genes in CD4+ T cells from E6/E7-vaccinated donors had differential effects on DLI-induced anti-tumor immunity (FIG. 2D). Interestingly, tumor-bearing recipients of vaccinated CD4+ T cells containing deletion of either PD-1 or TGFβR2 had slightly lower tumor-free survival than recipients of genetically unmodified CD4+ T cells and had significantly worse overall survival than recipients of cells with deletion of either ITK or foxp3 (FIG. 2D and Table 16).

TABLE 16
Effect of vaccinating the cell and deleting genes in vaccinated donor
CD4+ T cells on anti-tumor efficacy of donor lymphocyte infusion

| | | Median | | |
|---|---|---|---|---|
| | | tumor-free | Median severe | |
| | Gene | survival | disease-free | Median survival |
| Vaccine | Knockout | (days) | survival (days) | (days) |
| − | − | 31.5 | 54.5 | 56 |
| + | Cas9* | 39.5 | 54.5 | Not reached |
| + | PD-1 | 37 | 47 | 56 |
| + | TGFBR2 | 37 | 47 | 53 |
| + | SOCS1 | 39 | 56 | Not reached |
| + | Foxp3 | 47 | Not reached | Not reached |
| + | ITK | 56 | Not reached | Not reached |

These results indicate that CRISPR-mediated deletion of genes involved in CD4+ T cell differentiation and function can impact the anti-tumor efficacy of cell therapy from healthy donors vaccinated against tumor antigens-ITK or FOXP3 deletion appear to be beneficial, whereas PDCD1 or TGFBR2 deletion results in lower tumor-free survival compared to genetically unmodified CD4+ T cells.

### Example 11. Additional Gene Deletions on the Anti-Tumor Effects of CD4+ T Cells from MHC-Haploidentical Donors Vaccinated Against a Tumor Antigen

BALB/c×C57BL/6 (CB6 F1) donors will be vaccinated weekly for three doses with an intramuscular injection of 25 micrograms of pBI-11, a DNA vaccine encoding the E6 and E7 antigens of HPV16. One week after the third vaccine, CD4+ T cells will be enriched to near purity using immunomagnetic beads and left untreated, transfected with a Cas9 nucleoprotein, or the Cas9 nucleoprotein plus guide RNAs to inactivate single genes: Bruton's tyrosine kinase (BTK), delta isoform of phosphoinositide 3-kinase (PI3Kδ), helios, blimp1, GATA3, IL-10, STAT3, TOX, CD25, Ezh2, LAG-3, TNF-alpha, or combinations thereof. After resting the cells overnight, 3 million unvaccinated, vaccinated but not nucleofected, vaccinated and Cas9 nucleofected, or single gene deficient CD4+ T cells will be co-injected with 5 million syngeneic (CB6 F1), CD3-depleted spleen cells into MHC-haploidentical C57BL/6×C3H (B6C3 F1) mice that had received 50,000 TC-1 tumor cells (expressing E6 and E7 of HPV16) 14 days earlier and cyclophosphamide 200 mg/kg intraperitoneally the day before infusion.

### Example 12. Combination Gene Deletions on the Anti-Tumor Effects of CD4+ T Cells from MHC-Haploidentical Donors Vaccinated Against a Tumor Antigen

BALB/c×C57BL/6 (CB6 F1) donors will be vaccinated weekly for three doses with an intramuscular injection of 25 micrograms of pBI-11, a DNA vaccine encoding the E6 and E7 antigens of HPV16. One week after the third vaccine, CD4+ T cells will be enriched to near purity using immunomagnetic beads and left untreated, transfected with a Cas9 nucleoprotein, or the Cas9 nucleoprotein plus guide RNAs to inactivate combinations of two or more genes: Bruton's tyrosine kinase (BTK), interleukin-2-inducible T cell kinase (ITK), delta isoform of phosphoinositide 3-kinase (PI3Kδ), helios, blimp1, SOCS1, GATA3, IL-10, STAT3, TGF-beta Receptor II, LAG-3, PD-1, TNF-alpha, TOX, CD25, foxp3, Ezh2. After resting the cells overnight, 3 million unvaccinated, vaccinated but not nucleofected, vaccinated and Cas9 nucleofected, or single gene deficient CD4+ T cells will be co-injected with 5 million syngeneic (CB6 F1), CD3-depleted spleen cells into MHC-haploidentical C57BL/6×C3H (B6C3 F1) mice that had received 50,000 TC-1 tumor cells (expressing E6 and E7 of HPV16) 14 days earlier and cyclophosphamide 200 mg/kg intraperitoneally the day before infusion.

Although the invention has been described with references to the above examples, it will be understood that modifications and variations are encompassed within the spirit and scope of the invention. Accordingly, the invention is limited only by the following claims.

1. A pharmaceutical composition comprising a plurality of isolated leukocytes that are obtained from a donor subject and are mismatched to a recipient subject for at least one human leukocyte antigen (HLA) Class II allele in the donor versus recipient (graft-versus-host) direction relative to the recipient subject,
wherein the leukocytes are depleted of CD8+ T cells by about 10-fold or greater relative to un-depleted leukocytes,
wherein the leukocytes are modified to suppress Bruton's tyrosine kinase (BTK), interleukin-2-inducible T cell kinase (ITK), delta isoform of phosphoinositide 3-kinase (PI3Kδ), helios, blimp1, SOCS1, GATA3, IL-10, STAT3, TOX, CD25, foxp3, Ezh2, TGF-beta Receptor II, LAG-3, PD-1, TNF-alpha, or combinations thereof.

2. A pharmaceutical composition comprising a plurality of isolated leukocytes obtained from an allogeneic donor subject, wherein the donor CD4+ T cells have been stimulated in vivo or ex vivo by an antigen present in a recipient subject, and the donor subject comprises at least one HLA Class II allele match relative to the recipient,
wherein the leukocytes are depleted of CD8+ T cells by about 10-fold or greater relative to un-depleted leukocytes,
wherein the leukocytes are modified to suppress Bruton's tyrosine kinase (BTK), interleukin-2-inducible T cell kinase (ITK), delta isoform of phosphoinositide 3-kinase (PI3Kδ), helios, blimp1, SOCS1, GATA3, IL-10, STAT3, TOX, CD25, foxp3, Ezh2, TGF-beta Receptor II, LAG-3, PD-1, TNF-alpha, or combinations thereof.

3. A pharmaceutical composition comprising a plurality of isolated leukocytes that are obtained from a donor subject and (i) are mismatched to a recipient subject for at least one HLA Class II allele mismatch in the donor versus recipient (graft-versus-host) direction relative to the recipient subject and (ii) the donor CD4+ T cells have been stimulated in vivo or ex vivo by an antigen present in a recipient subject, and the donor subject comprises at least one human leukocyte HLA Class II allele match relative to the recipient,
wherein the leukocytes are depleted of CD8+ T cells by about 10-fold or greater relative to un-depleted leukocytes,
wherein at least a portion of the CD4+ T cells are modified to inhibit the activity of Bruton's tyrosine kinase (BTK), interleukin-2-inducible T cell kinase (ITK), delta isoform of phosphoinositide 3-kinase (PI3Kδ), helios, blimp1, SOCS1, GATA3, IL-10, STAT3, TGF-beta Receptor II, LAG-3, PD-1, TNF-alpha, TOX, CD25, foxp3, Ezh2, or combinations thereof.

4. The pharmaceutical composition of claim 1, wherein at least a portion of the T cells are differentiated to Th1 CD4+ T cells.

5. The pharmaceutical composition of claim 1, wherein the T cells are biased toward Th1 CD4+ T cell differentiation by inhibition of one or more of BTK, ITK, PI3Kδ, Foxp3, GATA3, STAT3, CD25, or Ezh2.

6-13. (canceled)

14. The pharmaceutical composition of claim 1, wherein the T cells are biased toward Th1 CD4+ T cell differentiation and against regulatory T cell differentiation by inhibition of both BTK and PI3Kδ.

15. The pharmaceutical composition of claim 1, wherein the T cells are biased toward Th1 CD4+ T cell differentiation by inhibition and against regulatory T cell differentiation of both ITK and PI3Kδ.

16. The pharmaceutical composition of claim 3, wherein the T cells are biased toward Th1 CD4+ T cell differentiation by inhibition and against regulatory T cell differentiation of BTK, ITK and PI3Kδ.

17. The pharmaceutical composition of claim 1, wherein BTK, ITK and PI3Kδ, helios, blimp1, SOCS1, GATA3, IL-10, STAT3, TOX, CD25, TGF-beta Receptor II, LAG-3, PD-1, TNF-alpha, foxp3, or Ezh2 are inhibited with an inhibitor or by genetic modification.

18. The pharmaceutical composition of claim 1, wherein the BTK inhibitor is acalabrutinib, zanubrutinib, LFM-A13, dasatinib or AVL-292.

19. The pharmaceutical composition of claim 1, wherein the BTK inhibitor is not ibrutinib.

20. The pharmaceutical composition of claim 1, wherein the ITK inhibitor is aminothiazole, aminobenzimidazole, indole, pyridine or prn694.

21. The pharmaceutical composition of claim 1, wherein the PI3Kδ inhibitor is idelalisib, copanlisib, duvelisib, umbralisib, ME-4401, RP6503, perifosine, buparlisib, or dactolisib.

22. The pharmaceutical composition of claim 17, wherein the inhibitor is a small molecule, a small interfering RNA (siRNA), or short hairpin RNA (shRNA).

23. The pharmaceutical composition of claim 17, wherein BTK, ITK, PI3Kδ, Helios, Blimp1, SOCS1, Foxp3, GATA3, IL-10, TGF-beta Receptor II, LAG-3, PD-1, TNF-alpha, STAT3, Ezh2, CD25, or TOX are inhibited by deleting the BTK gene, the ITK gene, the PI3Kδ gene, the Helios gene, the Blimpi gene, the SOCS1 gene, the Foxp3 gene, the TGF-beta Receptor II gene, the LAG-3 gene, the PD-1 gene, the TNF-alpha gene, the GATA3 gene, the IL-10 gene, the STAT3 gene, the Ezh2 gene, the CD25 gene, or the TOX gene from the genome.

24. The pharmaceutical composition of claim 23, wherein the BTK gene, the ITK gene, the PI3Kδ gene, the Helios gene, the Blimp1 gene, the SOCS1 gene, the Foxp3 gene, the TGF-beta Receptor II gene, the LAG-3 gene, the PD-1 gene, the TNF-alpha gene, the GATA3 gene, the IL-10 gene, the STAT3 gene, the Ezh2 gene, the CD25 gene, or the TOX gene is deleted from the genome using CRISPR or TALEN.

25. (canceled)

26. The pharmaceutical composition of claim 1, wherein differentiation of the T cells into T regulatory cells is attenuated through inhibition of PI3Kδ, Foxp3, CD25, or Ezh2.

27. The pharmaceutical composition of claim 26, wherein PI3Kδ, Foxp3, CD25, or Ezh2 is inhibited with a PI3Kδ inhibitor, a Foxp3 inhibitor, a CD25 inhibitor, or a Ezh2 inhibitor or by genetic modification.

28. The pharmaceutical composition of claim 27, wherein the PI3Kδ inhibitor is idelalisib, copanlisib, duvelisib, umbralisib, ME-4401, RP6503, perifosine, buparlisib, or dactolisib.

29. The pharmaceutical composition of claim 27, wherein the genetic modification comprises deletion of the PI3Kδ gene, the Foxp3 gene, the CD25 gene, or the Ezh2 gene.

30. The pharmaceutical composition of claim 29, wherein differentiation of the T cells into T regulatory cells or the function of regulatory T cells is attenuated with a small molecule, a small interfering RNA (siRNA), or short hairpin RNA (shRNA) against BTK, ITK, PI3Kδ, Helios, Blimp1, SOCS1, Foxp3, GATA3, IL-10, STAT3, TGF-beta Receptor II, LAG-3, PD-1, TNF-alpha, Ezh2, CD25, or TOX.

31. The pharmaceutical composition of claim 27, wherein differentiation of the T cells into T regulatory cells or the function of T regulatory cells is attenuated by modifying the isolated leukocytes obtained from the donor subject to express a dominant negative transforming growth factor-beta RII receptor.

32. The pharmaceutical composition of claim 1, wherein differentiation of the T cells into Th2 cells or function as Th2 cells is attenuated with an inhibitor or by genetic modification.

33. The pharmaceutical composition of claim 32, wherein differentiation of the T cells into Th2 cells or function as Th2 cells is attenuated through inhibition of GATA3.

34. The pharmaceutical composition of claim 1, wherein differentiation of the T cells into Th17 cells or function as Th17 cells is attenuated through inhibition of STAT3.

35. The pharmaceutical composition of claim 2, wherein the HLA Class II match is an HLA-DRB1 allele, an HLA-DQB1 allele, or an HLA-DPB1 allele.

36. The pharmaceutical composition of claim 1, wherein activation of myeloid cells is inhibited.

37. The pharmaceutical composition of claim 36, wherein activation of myeloid cells is inhibited through inhibition of BTK, ITK, PI3Kδ, Helios, Blimp1, SOCS1, Foxp3, TGF-beta Receptor II, LAG-3, PD-1, TNF-alpha, GATA3, IL-10, STAT3, or TOX.

38-113. (canceled)
